(19) United States
(12) Patent Application Publication
(10) Pub. No.: US 2023/0368861 A1
(43) Pub. Date: Nov. 16, 2023

(54) MACHINE LEARNING TECHNIQUES FOR PREDICTING THERMOSTABILITY

(71) Applicant: Amgen Inc., Thousand Oaks, CA (US)

(72) Inventors: Ameya Harmalkar, Baltimore, MD (US); Kathy Yufeng Wei, Berkeley, CA (US)

(73) Assignee: Amgen Inc., Thousand Oaks, CA (US)

(21) Appl. No.: 18/195,155

(22) Filed: May 9, 2023

Related U.S. Application Data
(60) Provisional application No. 63/340,332, filed on May 10, 2022.

(51) Int. Cl. G16B 15/20; G16B 30/00; G16B 40/20

(52) U.S. Cl. CPC G16B 15/20; G16B 30/00; G16B 40/20

(57) ABSTRACT

Techniques for computationally screening a set of single-chain variable fragments (scFvs). The techniques include determining, using a machine learning model, a thermostability indication for each scFv in a set of scFvs to obtain a plurality of thermostability indications, the set of scFvs comprising a first scFv having a first residue sequence, the determining comprising: obtaining, using information indicative of a 3D structure of the first scFv, interaction energy metrics for each of a plurality of pairs of residues in the first residue sequence; generating a first set of features using the interaction energy metrics; and providing the first set of features as input to the machine learning model to obtain a corresponding output indicative of a first thermostability for the first scFv; identifying a subset of the set of scFvs for subsequent production based on the plurality of thermostability indications; and producing at least one of the identified scFvs.

### CROSS REFERENCE TO RELATED APPLICATIONS

The present application claims the benefit of priority under 35 U.S.C. § 119(e) of U.S. Provisional Patent Application Ser. No. 63/340,332 filed on May 10, 2022, under Attorney Docket No. A1350.70001US00, and entitled “MACHINE LEARNING TECHNIQUES FOR PREDICTING SINGLE-CHAIN VARIABLE FRAGMENT (SCFV) THERMOSTABILITY,” which is herein incorporated by reference in its entirety.

### BACKGROUND

Monoclonal antibodies (mAbs) represent a large class of therapeutic agents, with more than 100 FDA-approved products marketed in the US. Multi-specific biologics engaging more than one target or epitope on the same target are of growing importance for accessing novel, therapeutically-relevant pathways, and mechanisms of action. Several multi-specific biologics are approved for use and many more are in clinical and preclinical development.

A common building block for the construction of multi-specific biologics is the single-chain variable fragment (scFv), consisting of the target-engaging antibody variable heavy chain (VH) linked to the variable light chain (VL) via a flexible linker. Multi-specific format platforms such as the BiTE, IgG-scFv, and XmAb incorporate scFv modules.

### SUMMARY

Some embodiments provide for a method for computationally screening a set of single-chain variable fragments (scFvs) based on thermostability of the scFvs predicted by a trained machine learning model, the set of scFvs comprising scFvs having different residue sequences, the method comprising: determining, using the trained machine learning model and at least one computer hardware processor, a thermostability indication for each scFv in the set of scFvs to obtain a plurality of thermostability indications, the set of scFvs comprising a first scFv having a first residue sequence, the determining comprising: obtaining, using information indicative of a three-dimensional (3D) structure of the first scFv, interaction energy metrics for each of a plurality of pairs of residues, the residues being in the first residue sequence; generating a first set of features to provide as input to the trained machine learning model, the generating comprising including the interaction energy metrics in the first set of features; and providing the first set of features as input to the trained machine learning model to obtain a corresponding output indicative of a first thermostability for the first scFv; identifying a subset of the set of scFvs for subsequent production based on the plurality of thermostability indications; and producing at least one of the scFvs in the identified subset.

In some embodiments, the set of scFvs further comprises a second scFv different from the first scFv, the second scFv having a second residue sequence. In some embodiments, determining the thermostability indication for each scFv in the set of scFvs further comprises: obtaining second interaction energy metrics for each of a second plurality of pairs of second residues, the second residues being in the second residue sequence; generating a second set of features to provide as input to the trained machine learning model, the generating comprising including the second interaction energy metrics in the second set of features; and providing the second set of features as input to the trained machine learning model to obtain a corresponding output indicative of a second thermostability for the second scFv.

In some embodiments, the output indicative of the first thermostability for the first scFv indicates a first temperature at which the first scFv is thermostable.

In some embodiments, the first temperature is an estimate of a temperature corresponding to half maximal binding of the first scFv.

In some embodiments, the output indicative of the first thermostability for the first scFv indicates a first temperature range including at least one temperature at which the first scFv is thermostable.

In some embodiments, the first temperature range is an estimate of a temperature range that includes a temperature corresponding to half maximal binding of the first scFv.

In some embodiments, providing the first set of features as input to the trained machine learning model to obtain the output indicative of the first thermostability for the first scFv comprises: classifying, using the trained machine learning model, the first scFv into one of a plurality of classes using the first set of features, wherein each of the plurality of classes corresponds to a respective temperature range.

In some embodiments, obtaining the interaction energy metrics comprises: determining the information indicative of the 3D structure of the first scFv by using protein structure prediction software to generate the information indicative of the 3D structure from the first residue sequence.

In some embodiments, obtaining the interaction energy metrics comprises: determining the interaction energy metrics using molecular modeling software to generate the interaction energy metrics using the information indicative of the 3D structure of the first scFv.

In some embodiments, generating the first set of features comprises: for each particular energy metric of the interaction energy metrics, generating a respective two-dimensional (2D) matrix of values of the particular energy metric, wherein rows and columns of the 2D matrix correspond to respective residues in the first residue sequence, and wherein an entry in the ith row and jth column of the 2D matrix corresponds to a value of the particular energy metric for the ith residue in the first residue sequence and the jth residue in the first residue sequence; and including the generated 2D matrix in the first set of features.

In some embodiments, the generated 2D matrix includes a row for at least 75% of the residues in the first residue sequence. In some embodiments, the generated 2D matrix includes a row for at least 90% of the residues in the first residue sequence. In some embodiments, the generated 2D matrix includes a row for at least 95% of the residues in the first residue sequence. In some embodiments, the generated 2D matrix includes a row for at least 99% of the residues in the first residue sequence.

In some embodiments, the generated 2D matrix includes a row for each residue in the first residue sequence.

In some embodiments, generating the first set of features further comprises: encoding the first residue sequence to obtain an encoded sequence; and including the encoded sequence in the first set of features.

In some embodiments, encoding the first residue sequence comprises: one-hot-encoding the first residue sequence to obtain the encoded sequence, the encoded sequence comprising a one-hot-encoded version of the first residue sequence.

In some embodiments, the trained machine learning model comprises a trained neural network model.

In some embodiments, the trained neural network model comprises a trained convolutional neural network (CNN) model, the trained CNN model having a plurality of 2D convolutional layers.

In some embodiments, the trained CNN model further comprises a fully connected layer.

In some embodiments, the trained CNN model is configured to output a plurality of probabilities that an scFv is thermostable in each of a plurality of temperature ranges.

In some embodiments, providing the first set of features as input to the trained machine learning model to obtain the corresponding output indicative of the first thermostability for the first scFv comprises: providing the first set of features to the trained CNN model to obtain a first plurality of probabilities that the first scFv is thermostable in each of the plurality of temperature ranges; and determining the first thermostability as either: (i) a temperature range in the plurality of temperature ranges associated with the highest probability in the first plurality of probabilities; or (ii) a temperature determined as a weighted linear combination of mean values of the temperature ranges weighted by the probabilities in the first set of probabilities.

In some embodiments, identifying the subset of the set of scFvs for subsequent production based on the plurality of determined thermostability indications comprises: determining whether the first thermostability for the first scFv satisfies at least one criterion; and after determining that the first thermostability satisfies the at least one criterion, identifying the first scFv for subsequent production.

Some embodiments further comprise testing the thermostability of the at least one of the scFvs in an in vitro assay.

Some embodiments provide for a method for predicting thermostability of a single-chain variable fragments (scFv) using a trained machine learning model, the method comprising: determining, using the trained machine learning model and at least one computer hardware processor, a first thermostability indication for a first scFv, the first scFv having a first residue sequence: obtaining, using information indicative of a three-dimensional (3D) structure of the first scFv, interaction energy metrics for each of a plurality of pairs of residues, the residues being in the first residue sequence; generating a first set of features to provide as input to the trained machine learning model, the generating comprising including the interaction energy metrics in the first set of features; and providing the first set of features as input to the trained machine learning model to obtain a corresponding output indicative of a first thermostability for the first scFv.

Some embodiments provide for a system, comprising: at least one computer hardware processor; and at least one non-transitory computer-readable storage medium storing processor-executable instructions that, when executed by the at least one computer hardware processor, cause the at least one computer-hardware processor to perform a method for predicting thermostability of a single-chain variable fragments (scFv) using a trained machine learning model, the method comprising: determining, using the trained machine learning model and at least one computer hardware processor, a first thermostability indication for a first scFv, the first scFv having a first residue sequence: obtaining, using information indicative of a three-dimensional (3D) structure of the first scFv, interaction energy metrics for each of a plurality of pairs of residues, the residues being in the first residue sequence; generating a first set of features to provide as input to the trained machine learning model, the generating comprising including the interaction energy metrics in the first set of features; and providing the first set of features as input to the trained machine learning model to obtain a corresponding output indicative of a first thermostability for the first scFv.

Some embodiments provide for at least one non-transitory computer-readable storage medium storing processor-executable instruction that, when executed by at least one computer hardware processor, cause the at least one computer hardware processor to perform a method for predicting thermostability of a single-chain variable fragments (scFv) using a trained machine learning model, the method comprising: determining, using the trained machine learning model and at least one computer hardware processor, a first thermostability indication for a first scFv, the first scFv having a first residue sequence: obtaining, using information indicative of a three-dimensional (3D) structure of the first scFv, interaction energy metrics for each of a plurality of pairs of residues, the residues being in the first residue sequence; generating a first set of features to provide as input to the trained machine learning model, the generating comprising including the interaction energy metrics in the first set of features; and providing the first set of features as input to the trained machine learning model to obtain a corresponding output indicative of a first thermostability for the first scFv.

Some embodiments further comprise determining, using the trained machine learning model and the at least one computer hardware processor, a thermostability indication for each scFv in a set of scFvs to obtain a plurality of thermostability indications, the set of scFvs comprising the first scFv.

Some embodiments further comprise identifying a subset of the set of scFvs for subsequent production based on the plurality of thermostability indications.

Some embodiments provide for a method for computationally screening a set of monoclonal antibodies (mAbs) based on thermostability of the mAbs predicted by a trained machine learning model, the set of mAbs comprising mAbs having different residue sequences, the method comprising: determining, using the trained machine learning model and at least one computer hardware processor, a thermostability indication for each mAb in the set of mAbs to obtain a plurality of thermostability indications, the set of mAbs comprising a first mAb having a first residue sequence, the determining comprising: obtaining, using information indicative of a three-dimensional (3D) structure of the first mAb, interaction energy metrics for each of a plurality of pairs of residues, the residues being in the first residue sequence; generating a first set of features to provide as input to the trained machine learning model, the generating comprising including the interaction energy metrics in the first set of features; and providing the first set of features as input to the trained machine learning model to obtain a corresponding output indicative of a first thermostability for the first mAb; identifying a subset of the set of mAbs for subsequent production based on the plurality of thermostability indications; and producing at least one of the mAbs in the identified subset.

Some embodiments further comprise testing the thermostability of the at least one of the mAbs in an in vitro assay.

Some embodiments provide for a method for computationally screening a set of antibodies based on thermostability of the antibodies predicted by a trained machine learning model, the set of antibodies comprising antibodies having different residue sequences, the method comprising: determining, using the trained machine learning model and at least one computer hardware processor, a thermostability indication for each antibody in the set of antibodies to obtain a plurality of thermostability indications, the set of antibodies comprising a first antibody having a first residue sequence, the determining comprising: obtaining, using information indicative of a three-dimensional (3D) structure of the first antibody, interaction energy metrics for each of a plurality of pairs of residues, the residues being in the first residue sequence; generating a first set of features to provide as input to the trained machine learning model, the generating comprising including the interaction energy metrics in the first set of features; and providing the first set of features as input to the trained machine learning model to obtain a corresponding output indicative of a first thermostability for the first antibody; identifying a subset of the set of antibodies for subsequent production based on the plurality of thermostability indications; and producing at least one of the antibodies in the identified subset.

Some embodiments further comprise testing the thermostability of the at least one of the antibodies in an in vitro assay.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a diagram depicting an illustrative technique 100 for determining thermostability of a single-chain variable fragment (scFv), in accordance with some embodiments of the technology described herein.

FIG. 1B is a block diagram of an example system 150 for determining thermostability of scFvs, in accordance with some embodiments of the technology described herein.

FIG. 2A is a flowchart of an illustrative process 200 for computationally screening a set of scFvs in order to identify a subset of the set of scFvs to produce, in accordance with some embodiments of the technology described herein.

FIG. 2B is a flowchart of an illustrative process 250 for determining thermostability for an scFv, in accordance with some embodiments of the technology described herein.

FIG. 3A is a diagram of an illustrative technique for computationally screening a set of scFvs using a trained machine learning model that is trained to generate a thermostability indication for an scFv from input containing interaction energy metrics of pairs of residues in the scFv, in accordance with some embodiments of the technology described herein.

FIG. 3B is a diagram of an illustrative technique for computationally screening a set of scFvs using a trained machine learning model that is trained to generate a thermostability indication for an scFv from input containing interaction energy metrics of pairs of residues in the scFv and features representing the sequence of the scFv, in accordance with some embodiments of the technology described herein.

FIG. 3C is a diagram of an illustrative technique for computationally screening a set of scFvs using a trained machine learning model that is trained to generate a thermostability indication for an scFv from input containing features representing the sequence of the scFv, in accordance with some embodiments of the technology described herein.

FIG. 4A is a flowchart of an illustrative process 400 for training a machine learning model to predict thermostability of an scFv, in accordance with some embodiments of the technology described herein.

FIG. 4B is a diagram of an example technique 450 for experimentally generating data used to train a model to predict thermostability of an scFv, in accordance with some embodiments of the technology described herein.

FIG. 5A is a flowchart of an illustrative process 500 for computationally screening a set of antibodies in order to identify a subset of the set of antibodies to produce, in accordance with some embodiments of the technology described herein.

FIG. 5B is a flowchart of an illustrative process 550 for determining thermostability for an antibody, in accordance with some embodiments of the technology described herein.

FIG. 6 is a diagram of an example of an scFv, a set of features generated for an scFv, and a machine learning model trained to predict thermostability of an scFv, in accordance with some embodiments of the technology described herein.

FIG. 7 is a diagram of an example technique for training and using the trained machine learning models to predict thermostability of scFvs, in accordance with some embodiments of the technology described herein.

FIG. 8 is a diagram of an example technique 800 for predicting thermostability of an scFv using a trained convolutional neural network (CNN) model, in accordance with some embodiments of the technology described herein.

FIG. 9 shows t-stochastic neighbor embeddings (t-SNE) showing improved performance of the techniques described herein for predicting thermostability of scFvs using a machine learning model trained using interaction energy metrics of scFvs, in accordance with some embodiments of the technology described herein.

FIG. 10 shows a receiver-operating-characteristic (ROC) curve showing the performance of a machine learning model trained to predict thermostability of scFvs using interaction energy metrics for pairs of residues of scFv residue sequences, in accordance with some embodiments of the technology described herein.

FIG. 11 shows a graph comparing performance of the various machine learning models described herein for predicting thermostability of scFvs, in accordance with some embodiments of the technology described herein.

FIG. 12A contains graphs showing thermostability prediction performance using various neural network architectures, in accordance with some embodiments of the technology described herein.

FIG. 12B is a table showing that prediction of thermostability using an ensemble of neural network models is more accurate than prediction of thermostability using a single neural network model, in accordance with some embodiments of the technology described herein.

FIG. 12C shows t-SNE showing improve performance of the techniques described herein for predicting thermostability of scFvs using an ensemble of machine learning models, in accordance with some embodiments of the technology described herein.

FIGS. 13A-13B are graphs showing that thermostability predictions using the machine learning techniques, in accordance with some embodiments of the technology described herein, can be used to observe trends in thermostability of scFvs.

FIG. 14 is a diagram depicting an example technique for predicting thermostability of an scFv using a trained language model, in accordance with some embodiments of the technology described herein.

FIG. 15A is a diagram of an example pre-trained language model configured to make zero-shot thermostability predictions using sequence data, in accordance with some embodiments of the technology described herein.

FIG. 15B is a diagram of an example fine-tuned pre-trained language model configured to make thermostability predictions using sequence data, in accordance with some embodiments of the technology described herein.

FIGS. 15C-15F are graphs showing that fine-tuned predictions achieve improved correlation with thermostability as compared to zero-shot predictions, in accordance with some embodiments described herein.

FIG. 16 is a diagram showing that the thermostability predictions determined using the machine learning techniques, in accordance with some embodiments of the technology described herein, agree with the experimentally-determined thermostabilities.

FIGS. 17A-17B show that thermostability predictions using the machine learning techniques, in accordance with some embodiments of the technology described herein, can be used to identify thermostable mutations as compared to conventional germ lining techniques.

FIGS. 18A-18B are graphs showing that thermostability predictions output by a supervised convolutional neural network achieve improved correlation with thermostability as compared to thermostability predictions by an unsupervised pre-trained language model, in accordance with some embodiments of the technology described herein.

FIG. 19 contains graphs comparing performance of the various machine learning models described herein for predicting thermostability of scFvs, in accordance with some embodiments of the technology described herein.

FIG. 20 contains graphs showing that training and prediction based on residue-pair interaction energy metrics is more accurate than training and prediction based on encoded sequences and training and prediction based on both encoded sequences and residue-pair interaction energy metrics, in accordance with some embodiments of the technology described herein.

FIGS. 21A-21B are graphs showing that training and prediction based on residue-pair interaction energy metrics is more accurate than training and prediction based on residue-pair interaction energy metrics and encoded sequences, in accordance with some embodiments of the technology described herein.

FIGS. 22A-22B show a distribution of training, validation, and test datasets, in accordance with some embodiments of the technology described herein.

FIG. 23 is a graph showing the temperature distribution of TS50 measurements of the experimental dataset, in accordance with some embodiments of the technology described herein.

FIG. 24 is a schematic diagram of an illustrative computing device with which aspects described herein may be implemented.

### DETAILED DESCRIPTION

The inventors have developed machine learning techniques for predicting thermostability for single-chain variable fragments (scFvs) or other multi-specific constructs. In some embodiments, predicting thermostability for an scFv includes processing a set of features generated for the scFv using a trained machine learning model. In some embodiments, the set of features is generated using a residue sequence of the scFv. For example, in some embodiments, the set of features includes interaction energy metrics for residue pairs in the residue sequence. Additionally, or alternatively, the set of features includes the residue sequence of the scFv. In some embodiments, the set of features is provided as input to a trained machine learning model to obtain an output indicative of a temperature at which the scFv is thermostable (e.g., a thermostability indication).

In some embodiments, the techniques described herein are used to screen a set of scFvs. For example, the set of scFvs may include one or more scFvs that are candidates for subsequent production. In some embodiments, the techniques for screening the set of scFvs include determining a thermostability indication for each scFv included in the set of scFvs and identifying a subset of the set of scFvs based on the determined thermostability indications. For example, the identified subset of scFvs may include scFvs that are thermostable. Thus, one or more of the scFvs included in the identified subset of scFvs may be subsequently produced (e.g., manufactured).

As described above, multi-specific biologics are of growing importance for accessing novel, therapeutically-relevant pathways and mechanisms of action. One property that is used to gauge the potential developability of a multi-specific biologic, scFv module or scFv-containing multi-specific is thermostability. Thermostability is a characteristic that may be indicative of its stability under certain environmental conditions. Various factors such as the particular amino acid sequence and/or the resulting structure of the scFv may impact the thermostability of a scFv. For example, interactions between amino acid residues, such as hydrophobic and electrostatic interactions, may influence the thermostability of an scFv. Additionally, or alternatively, the presence of particular bonds, such as disulfide bonds, may influence the thermostability of an scFv.

Thermostability is relevant because thermostable scFvs can withstand certain environmental conditions and have corresponding adaptations to preserve the scFv function under those conditions. For example, such thermostable scFvs may withstand exposure to a temperature in a range of temperatures that may occur in practice. By contrast, scFvs with poor thermostability properties may unfold and denature, resulting in loss of enzymatic activity, in temperature ranges which can realistically occur in practice.

Conventional approaches to optimizing scFvs for thermostability involve experimentally screening scFv candidates for thermostability. However, experimental screening is resource intensive, time consuming, and expensive because it requires producing and performing experiments on each scFv in a large set of candidate scFvs being screened to determine which scFvs have desired thermostability properties.

The inventors have appreciated that it would be especially useful to computationally screen scFvs for thermostability. However, the inventors recognized that conventional computational methods for predicting scFv thermostability are unreliable and inaccurate. For example, some conventional computational techniques include processing an amino acid sequence using a machine learning model trained on other amino acid sequences and their corresponding thermostabilities. The machine learning model is trained to predict thermostability for the amino acid sequence by identifying similar amino acid sequences that were used to train the model. However, while two amino acid sequences may have high similarity, this does not mean that they will have similar thermostabilities. For example, two amino acids may be identical but for a single mutation. However, this mutation might cause a drastic change in thermostability. Accordingly, the prediction generated by such a machine learning model may be inaccurate.

Other conventional computational techniques have been used to predict thermostability based on the total energy of a protein or fragment. However, such techniques still result in inaccurate predictions because they do not account for the structure of the scFv, which strongly influences its thermostability. By not accounting for structure of an scFv, the conventional techniques discount information that could drastically change the resulting thermostability prediction. Accordingly, these techniques are also unreliable and inaccurate.

The inventors have recognized a need for accurate computational methods to predict scFv thermostability from primary amino acid sequences of scFv candidates and appreciated that such methods would guide thermostability engineering efforts and would be invaluable to multispecific drug development. In particular, the inventors have recognized that taking the structure (e.g., 2D and/or 3D structure) of an scFv into account leads to more accurate thermostability predictions as compared to conventional techniques. Specifically, the inventors have developed techniques that take per-residue interactions into account when predicting thermostability. These interactions capture information about scFv structure and allow the prediction technique (e.g., a machine learning technique) to take that structure into account.

Accordingly, the inventors have developed machine learning techniques for predicting thermostability for single-chain variable fragments (scFvs). In order to determine thermostability for a particular scFv, the techniques involve deriving features from the primary amino acid sequence of the particular scFv and providing those features as input to a trained machine learning model to produce a corresponding output indicative of a thermostability for the particular scFv (a “thermostability indication”). The output may be a measure of thermostability such as a temperature at which the scFv is thermostable (e.g., a temperature corresponding to a half maximal binding of the scFv) or a temperature range including such a temperature. In some embodiments, the features provided as input to the trained machine learning model include only energy features, for example, interaction energy metrics between pairs of residues in the residue sequence of the particular scFv. Additionally, or alternatively, in some embodiments, the features may include an encoding of the residue sequence.

One example application of the machine learning techniques for predicting thermostability of scFvs is to computationally screen scFvs prior to production to identify those scFvs that have favorable thermostability properties. Thus, the machine learning techniques may be used to determine a thermostability indication for each scFv included in a set of scFvs being computationally screened and to identify a subset of the set of scFvs based on the determined thermostability indications. At least some of the scFvs so identified may be subsequently produced.

Accordingly, some embodiments provide for a method for computationally screening a set of single-chain variable fragments (scFvs) based on thermostability of the scFvs predicted by a trained machine learning model, the set of scFvs comprising scFvs having different residue sequences, the method comprising: (A) determining, using the trained machine learning model and at least one computer hardware processor, a thermostability indication for each scFv in the set of scFvs to obtain a plurality of thermostability indications, the set of scFvs comprising a first scFv having a first residue sequence, the determining comprising: (i) obtaining, using information indicative of a three-dimensional (3D) structure of the first scFv, interaction energy metrics for each of a plurality of pairs of residues, the residues being in the first residue sequence; (ii) generating a first set of features to provide as input to the trained machine learning model, the generating comprising including the interaction energy metrics in the first set of features; and (iii) providing the first set of features as input to the trained machine learning model to obtain a corresponding output indicative of a first thermostability for the first scFv; (B) identifying a subset of the set of scFvs for subsequent production based on the plurality of thermostability indications; and (C) producing at least one of the scFvs in the identified subset.

In some embodiments, the output indicative of the first thermostability for the first scFv indicates a first temperature at which the first scFv is thermostable. The first temperature may be an estimate of a temperature corresponding to half maximal binding of the first scFv (this may be termed the “TS50” temperature). In this way, the machine learning model may be configured to operate as a regression model.

In some embodiments, the output indicative of the first thermostability for the first scFv indicates a first temperature range including at least one temperature at which the first scFv is thermostable. The first temperature range may be an estimate of a temperature range that includes a temperature corresponding to half maximal binding of the first scFv. In some embodiments, providing the first set of features as input to the trained machine learning model to obtain the output indicative of the first thermostability for the first scFv comprises: classifying, using the trained machine learning model, the first scFv into one of a plurality of classes using the first set of features, wherein each of the plurality of classes corresponds to a respective temperature range. In this way, the machine learning model may be configured to operate as a classification model.

In some embodiments, the residue pair interaction energy metrics may be obtained in a two-stage process in which: (1) the residue sequence of the scFv is used to determine the information indicative of the 3D structure of the first scFv (e.g., using protein structure prediction software, examples of which are provided herein); and (2) determining the interaction energy metrics using the information indicative of the 3D structure of the first scFv. (e.g., by using molecular modeling software, examples of which are provided herein).

The inventors have recognized that the way in which interaction energy metrics are provided as input to a trained neural network model may influence that model's performance in predicting thermostability. In particular, the inventors recognized that organizing the interaction energy metrics in a two-dimensional array or matrix generates a matrix having local spatial structure amenable to analysis by convolutional neural network models and that organizing the interaction energy metrics in this manner may lead to improved performance in some embodiments (e.g., as opposed to providing a linear sequence interaction energy metrics).

Accordingly, in some embodiments, generating the first set of features comprises, for each particular energy metric, generating a respective two-dimensional matrix of values of the particular energy metric, wherein rows and columns of the 2D matrix correspond to respective residues in the first residue sequence. Thus, an entry in the ith row and jth column of the 2D matrix corresponds to a value of the particular energy metric between the ith residue in the first residue sequence and the jth residue in the first residue sequence.

While, in some embodiments, the interaction energy metrics between all pairs of residues of an scFv are provided as input to the trained machine learning model, in other embodiments, interaction energy metrics between only some pairs of residues are provided. For example, interaction energy metrics between at least 50%, at least 60%, at least 75%, at least 80%, at least 85%, at least 90%, at least 95%, or least 99% of the pairs of residues may be provided as input to the trained machine learning model. In some such embodiments, a 2D matrix of interaction energy metrics includes a row for at least 50%, 60%, 75%, 85%, 90%, 95%, and 99% of the residues in the residue sequence of the first scFv.

In some embodiments, sequence data may be provided as additional input (in addition to the interaction energy metrics) to the trained machine learning model. Accordingly, in some such embodiments, generating the first set of features further comprises encoding (e.g., one-hot encoding) the first residue sequence to obtain an encoded sequence; and including the encoded sequence in the first set of features.

In some embodiments, the trained machine learning model comprises a trained neural network model such as, for example, a convolutional neural network (CNN) model having a plurality of 2D convolutional layers. The CNN model may have a fully connected layer. Other types of architectures are also possible (e.g., having one or more dropout layer, one or more pooling layers, etc.).

In some embodiments, the trained CNN model is configured to output a plurality of probabilities that an scFv is thermostable in each of a plurality of temperature ranges. These probabilities may be used to either identify a single most likely temperature range (e.g., the temperature range associated with the largest probability value) or to determine an estimate of a particular temperature value, for example, as a weighted linear combination of mean values of the temperature ranges weighted by the respective probabilities of the temperature ranges.

As used herein, the terms “single-chain Fv,” or “scFv” refer to single polypeptide chain antibody fragments that comprise the variable regions from both the heavy and light chains but lack the constant regions. Generally, a single-chain Fv further comprises a peptide linker between the VH and VL domains, which enables it to form the desired structure which would allow for antigen binding. Such constructs are discussed in detail by Pluckthun in The Pharmacology of Monoclonal Antibodies, vol. 113, Rosenburg and Moore eds. Springer-Verlag, New York, pp. 269-315 (1994) and U.S. Pat. No. 7,112,324, titled “CD 19xCD3 SPECIFIC POLYPEPTIDES AND USES THEREOF”, each of which is incorporated by reference herein in its entirety. Structures that may be formed by scFvs include structures in a bispecific T cell engager (BiTE) format described in U.S. Pat. No. 7,112,324, titled “CD 19xCD3 SPECIFIC POLYPEPTIDES AND USES THEREOF”, (a fusion protein consisting of two single-chain variable fragments (scFvs) joined by a peptide linker), which is incorporated by reference herein in its entirety, with a variable heavy chain (VH) domain followed by a variable light chain (VL) domain, starting from the N-Terminal. The term “peptide linker” refers to an amino acid sequence by which the amino acid sequences of one (variable and/or binding) domain and another (variable and/or binding) domain of the scFv are linked with each other. Among the suitable peptide linkers are those described in U.S. Pat. No. 4,751,180, titled “EXPRESSION USING FUSED GENES PROVIDING FOR PROTEIN PRODUCT”, and U.S. Pat. No. 4,935,233, titled “COVALENTLY LINKED POLYPEPTIDE CELL MODULATORS”, or WO 88/09344, titled “TARGETED MULTIFUNCTIONAL PROTEINS”, each of which is incorporated by reference herein in its entirety.

It should be appreciated that the techniques described herein may be applied to predicting thermostability of scFvs as well as to any antibody construct having the variable domains (VH and VL domains) including constructs with or constructs without a peptide linker. Additionally, the techniques described herein may be applied to multi-specific constructs by utilizing a training dataset of multi-specific constructs having the same (or very similar, for example, at least 80%, 85%, 90%, 95%, 99% similarity) sequence and/or structure. Moreover, the techniques described herein may be applied to any type of protein by utilizing a training dataset of proteins having the same (or very similar, for example, at least 80%, 85%, 90%, 95%, 99% similarity) sequence and/or structure.

The term “multi-specific construct” refers to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof. The definition of the term “multi-specific construct” includes monovalent, bivalent and polyvalent/multivalent constructs and, thus, bispecific constructs, specifically binding to only two antigenic structures, as well as poly-specific/multi-specific constructs, which specifically bind more than two antigenic structures, e.g., three, four or more, through distinct binding domains. Moreover, the definition of the term “multi-specific construct” includes molecules consisting of only one polypeptide chain such as a scFv, as well as molecules consisting of more than one polypeptide chain, which chains can be either identical (homodimers, homotrimers or homo oligomers) or different (heterodimer, heterotrimer or heterooligomer). Examples for the above identified molecules and variants or derivatives thereof are described inter alia in Harlow and Lane, Antibodies a laboratory manual, CSHL Press (1988) and Using Antibodies: a laboratory manual, CSHL Press (1999), Kontermann and Dübel, Antibody Engineering, Springer, 2nd ed. 2010 and Little, Recombinant Antibodies for Immunotherapy, Cambridge University Press 2009, each of which is incorporated by reference herein in its entirety. Additional examples for the format of multi-specific constructs include a Fab fragment, a monovalent fragment having the VL, VH, CL and CH1 domains, a F(ab′)₂ fragment, a bivalent fragment having two Fab fragments linked by a disulfide bridge at the hinge region, an Fd fragment having the two VH and CH1 domains, an Fv fragment having the VL and VH domains of a single arm of an antibody, a dAb fragment (Ward et al., (1989) Nature 341:544-546, incorporated by reference herein in its entirety), which has a VH domain, an isolated complementarity determining region (CDR), and a single chain Fv (scFv). Examples of multi-specific constructs are e.g. described in WO 00/006605, titled “HETEROMINIBODIES”, WO 2005/040220, titled “MULTISPECIFIC DEIMMUNIZED CD3-BINDERS”, WO 2008/119567, titled “CROSS-SPECIES-SPECIFIC CD3-EPSILON BINDING DOMAIN”, WO 2010/037838, titled “CROSS-SPECIES-SPECIFIC SINGLE DOMAIN BISPECIFIC SINGLE CHAIN ANTIBODY”, WO 2013/026837, titled “BISPECIFIC T CELL ACTIVATING ANTIGEN BINDING MOLECULES”, WO 2013/026833, titled “BISPECIFIC T CELL ACTIVATING ANTIGEN BINDING MOLECULES”, US 2014/0308285, titled “HETERODIMERIC BISPECIFIC ANTIBODIES”, US 2014/0302037, titled “BISPECIFIC-FC MOLECULES”, WO 2014/144722, titled “BISPECIFIC FC MOLECULES”, WO 2014/151910, titled “HETERODIMERIC BISPECIFIC ANTIBODIES”, and WO 2015/048272, titled “V-C-FC-V-C ANTIBODY”, each of which is incorporated by reference herein in its entirety. Further, multi-specific constructs can be fragments of full-length antibodies, such as VH, VHH, VL, (s)dAb, Fv, Fd, Fab, Fab′, F(ab′)₂ or “r IgG” (“half antibody”).

Additionally, it should be appreciated that the techniques described herein may be applied to constructs that comprise modified fragments of antibodies, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multi-bodies” such as triabodies or tetrabodies, single domain antibodies such as nanobodies, or single variable domain antibodies comprising merely one variable domain, which might be VHH, VH or VL, that specifically bind an antigen or epitope independently of other V regions or domains, and Human Heavy-Chain Antibodies UniAb®, UniDab®, as described in WO2020206330A1, titled “HEAVY CHAIN ANTIBODIES BINDING TO PSMA”, which is incorporated by reference herein in its entirety.

The term “binding domain” refers to a domain which (specifically) binds to/interacts with/recognizes a given target epitope or a given target side on the target molecules (antigens), e.g., CD33 and CD3, respectively. The structure and function of the first binding domain (recognizing e.g. CD33), and preferably also the structure and/or function of the second binding domain (recognizing CD3), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Preferably the binding domain is characterized by the presence of three heavy chain CDRs (i.e., CDR1, CDR2 and CDR3 of the VH region) followed by three light chain CDRs (i.e., CDR1, CDR2 and CDR3 of the VL region). The binding domain of the multi-specific construct and the binding domain used in the training set should be consistently applied.

Thus, as should be appreciated from the foregoing, the techniques described herein are not limited to being applied to single-chain variable fragments and may also be applied to other constructs described herein. For example, the techniques described herein can be applied to monoclonal antibodies (mAbs). For example, a mAb sequence may be input as a VH sequence followed directly by the VL sequence (without an intervening linker sequence). As another example, a mAb sequence may be input as a VL sequence followed directly by the VH sequence (without an intervening linker sequence). The input sequence may be used to predict structure, which in turn may be used to calculate energy metrics. Then the energy metrics (and, optionally, an encoding of the input sequence) may be provided as input to the trained machine learning model to obtain a thermostability indication for the mAb. As another example, the techniques described herein may be applied to one or more types of multi-specific constructs, examples of which are provided herein.

In another example, the techniques described herein can be applied to any types of antibodies. For example, an antibody sequence may be used to predict structure, which in turn may be used to calculate energy metrics. Then the energy metrics (and, optionally, an encoding of the input sequence) may be provided as input to the trained machine learning model to obtain a thermostability indication for the antibody. In yet another example, the techniques described herein can be applied to any type of protein. For example, a protein sequence may be used to predict structure, which in turn may be used to calculate energy metrics. Then the energy metrics (and, optionally, an encoding of the input sequence) may be provided as input to the trained machine learning model to obtain a thermostability indication for the protein.

The techniques described herein may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination.

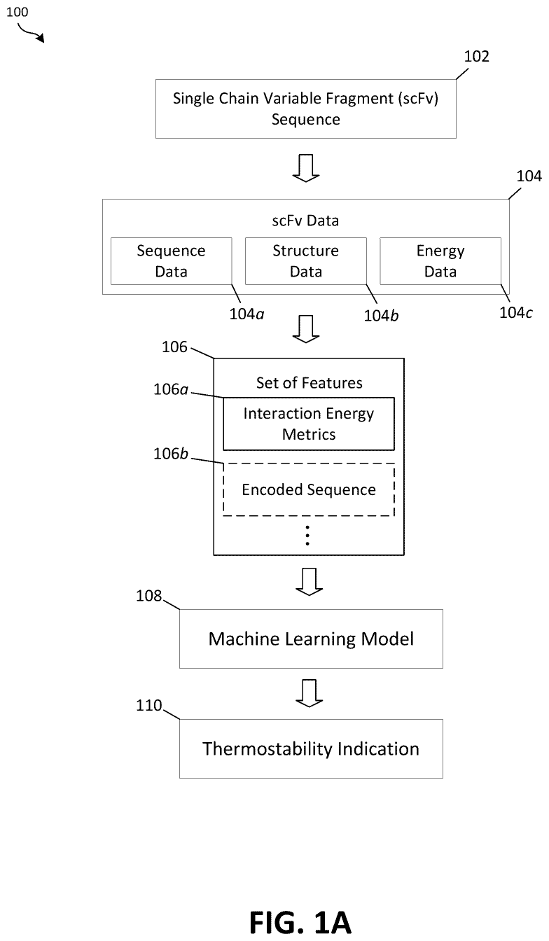

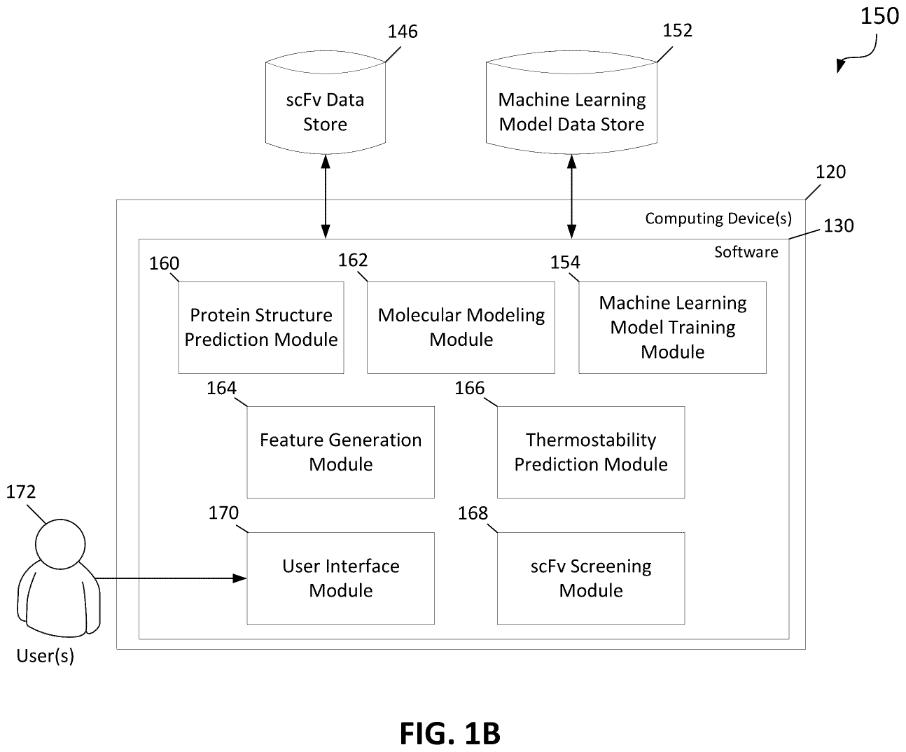

FIG. 1A is a diagram of an illustrative technique 100 for determining a thermostability indication 110 for a single-chain variable fragment (scFv) by providing features 106, generated from the residue sequence 102 of the scFv, as input to a machine learning model 108.

In some embodiments, the scFv sequence 102 specifies a residue sequence (e.g., primary amino acid sequence) of an scFv. The scFv sequence may specify an amino acid sequence for the heavy and light chains of the scFv as well as for the linker peptide. The scFv sequence 102 may be of any suitable length. For example, the scFv sequence 102 may have between 200 and 300 residues, between 225 and 275 residues, or between 236 and 254 residues, or any other suitable range within these ranges. In some embodiments, the linker peptide may consist of 10-25 amino acids and the scFv sequence 102 may contain a subsequence of that length representing the amino acids in the scFv's linker.

In some embodiments, the scFv sequence 102 may be specified by a user. For example, a user may interact with a user interface of a computing device (e.g., computing device(s) 120 shown in FIG. 1B) to specify the sequence of amino acid residues for scFv sequence 102. In some embodiments, the scFv sequence 102 may be specified automatically by a computing device (e.g., computing device(s) 120). For example, the computing device may be programmed to generate the sequence of amino acids for scFv sequence 102 (e.g., using machine learning, by iteratively changing amino acids in a predetermined order, randomly, etc.). This may be useful when a computer is programmed to automatically generate a list of scFvs for computational screening, for example, by automatically generating mutations by substituting one or amino acids in a starting scFv sequence to obtain a candidate scFv sequence, which may be evaluated during screening (e.g., based on its predicted thermostability). In some embodiments, the scFv sequence 102 may be generated at least in part automatically (e.g., by programmatically varying one or more amino acids in a sequence) and at least in part manually (e.g., based on user input specifying one or more amino acids in the sequence). The scFv sequence 102 may be specified in any suitable format (e.g., FASTA), as aspects of the technology described herein is not limited in this respect.

Next, as part of technique 100, scFv data 104 is generated from the scFv sequence 102. The scFv data 104 may include any type of data generated from the scFv sequence 102. In the illustrative embodiment shown in FIG. 1A, the scFv data 104 includes sequence data 104a, structure data 104b, and energy data 104c, each of which is described below. However, it should be appreciated that this example is illustrative and that, in other embodiments, scFv data 104 may include any other suitable data generated from scFv sequence 102 in addition to or instead of the types of data shown in FIG. 1A.

In some embodiments, the sequence data 104a includes the scFv sequence 102 itself or a subsequence thereof. Additionally, the sequence data 104a may include information about the sequence (e.g., amino acid statistics, length, sequence identifier, and/or any other information associated with the sequence). In some embodiments, the sequence data 104a is stored in a text-based file, such as a FASTA file, and/or in any other suitable format, as aspects of the technology described herein are not limited in this respect.

In some embodiments, the structure data 104b includes information indicative of the three-dimensional structure of the scFv sequence 102. The information indicative of the 3D structure may include description and/or annotation of protein structures including atomic coordinates, secondary structure assignments, and/or atomic connectivity data. The structure data 104b may be in any suitable format (e.g., Protein Data Bank (PDB) file format, Crystallographic Information File (CIF) format, macromolecular crystallographic information file (mmCIF) format) describing the 3D structure of the scFv.

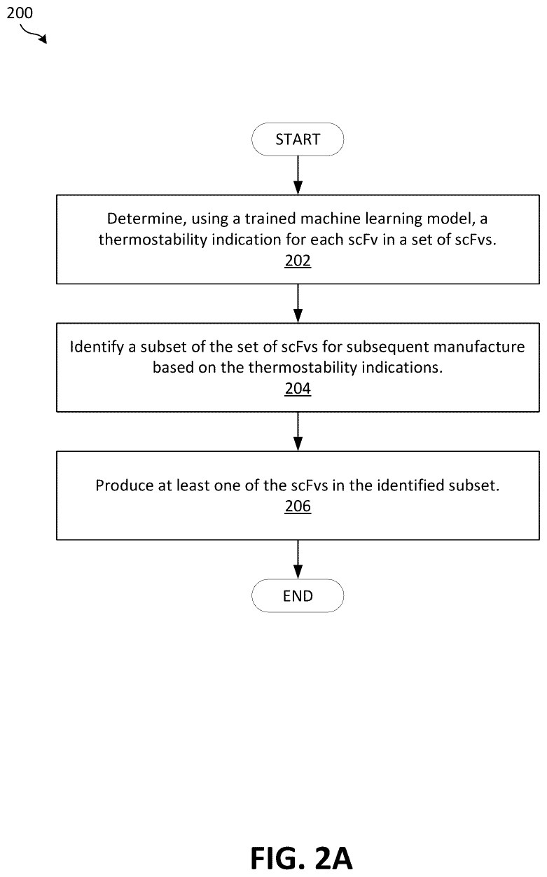

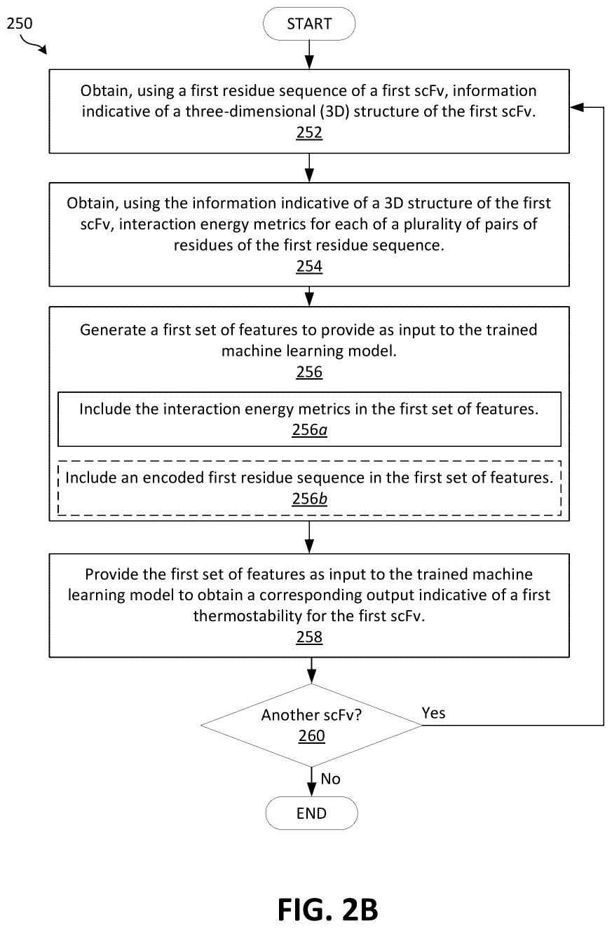

In some embodiments, the sequence data 104a is used to obtain the structure data 104b. For example, obtaining the structure data 104b may include processing the sequence data 104a using protein structure prediction software (e.g., protein structure prediction module 160 shown in FIG. 1B) to predict the 3D structure of the scFv. Techniques for obtaining information indicative of the 3D structure of an scFv are described herein including at least with respect to act 252 of process 250 shown in FIG. 2B.

In some embodiments, the energy data 104c includes information indicative of the energy levels of the scFv residue sequence 102 in its 3D conformation. For example, the energy data may include interaction energy metrics for one or more pairs of residues in the scFv residue sequence 102. Interaction energy metrics may include energies for several diverse types of interactions between a pair of residues. For example, the interaction energy metrics may account for energies of interactions between non-bonded atom pairs and statistical potentials used to describe backbone and side-chain torsional preferences in the scFv. In some embodiments, the energy data 104c is included in a delimited text file, such as a comma-separated value (CSV) file, for example.

In some embodiments, the structure data 104b is used to obtain the energy data 104c. For example, obtaining the energy data 104c may include processing the structure data 104b using molecular modeling software (e.g., using molecular modeling module 162 shown in FIG. 1B) to predict interaction energy metrics for each of multiple (e.g., some or all) pairs of residues of the scFv sequence 102. Techniques for obtaining interaction energy metrics are described herein including at least with respect to act 254 of process 250 shown in FIG. 2B.

After obtaining scFv data 104, the scFv data 104 is used to generate a set of features 106 for the scFv. In the embodiment of FIG. 1A, the set of features 106 includes interaction energy metrics 106a (e.g., included in energy data 104c). In some embodiments, the interaction energy metrics 106a include interaction energy metrics for each of multiple pairs of residues of the scFv sequence 102. The interaction energy metrics 106a may include interaction energy metrics for at least 75%, at least 80%, at least 85%, at least 90%, at least 95%, at least 98%, at least 99%, or all of the pairs of residues of the scFv sequence 102. In some other embodiments, the interaction energy metrics 106a may include interaction energy metrics for at most 99%, at most 98%, at most 95%, at most 90%, at most 85%, at most 80%, at most 75% or less of the pairs of residues of the scFv sequence 102. Thus, it should be appreciated, that interaction energy metrics 106a may include interaction energy metrics for a subset (i.e., not all) pairs of residues of the scFv sequence 102, in some embodiments.

As shown in FIG. 1A, the set of features 106 optionally includes an encoded sequence 106b for the scFv. The encoded sequence 106b may be an encoding of (at least a subsequence or all of) the scFv sequence 102. As one example, the encoded sequence 106b may have been obtained by one-hot encoding of scFv sequence 102. One-hot encoding is a technique for converting categorical data (each residue in the sequence is one of twenty possible amino acids) into numerical data (e.g., binary data, integer-valued data, real-valued data). The scFv sequence 102 may be one-hot encoded by being transformed into a series of 20-dimensional vectors, one for each amino acid in the sequence. Each coordinate of the vector may correspond to a respective one of 20 amino acids. Each amino acid may then be encoded into a single 20-dimensional vector having a 1 at the coordinate of that amino acid and 0s elsewhere. However, it should be appreciated that the aspects of technology described herein are not limited to using one-hot encoding to encode scFv sequences and that other methods for encoding categorical data may be used.

In another embodiment, the set of features 106 may include only encoded sequence data and not include any interaction energy metrics. Indeed, the machine learning techniques described herein may be used to predict thermostability for an scFv using energy features (e.g., interaction energy metrics) alone, using sequence features (e.g., using one-hot encoding of scFv sequence) alone, or using a combination of energy and sequence features. As described herein, different machine learning models may be used to perform the prediction depending on which features are utilized (e.g., 2D convolutional neural networks when the input includes interaction energy metrics and language models when it doesn't).

Aside from sequence and energy features, it should be appreciated that the set of features 106 may include one or more additional or alternative features, as aspects of the technology described herein are not limited in this respect. For example, one or more additional or alternative features may be obtained from scFv data 104 and included in the set of features 106.

After features 106 are generated, they are provided as input to a trained machine learning model 108 to obtain a corresponding thermostability indication 110 for the scFv. The machine learning model may be of any suitable type. For example, the machine learning model may be a neural network model, such as a convolutional neural network (CNN) model. The CNN model may have one or more convolutional layers (e.g., one or more two-dimensional convolutional layers). The CNN model may have a fully connected layer. As described herein, in some embodiments, a convolutional neural network model may be configured to receive as input only energy features (e.g., as a 1D or 2D matrix interaction energy metrics) or a combination of energy features and sequence features (e.g., a one-hot encoding of an scFv sequence).

As another example, the machine learning model may be a pre-trained language model adapted to the protein setting. For example, a bidirectional encoder representation from transformers (BERT) model, such as the ESM-1b or ESM-1v language model, may be used. These types of models are described in: A Rives, et al., Biological structure and function emerge from scaling unsupervised learning to 250 million protein sequences. Proc. Natl. Acad. Sci. 118 (2021), and J Meier, et al., Language models enable zero-shot prediction of the effects of mutations on protein function. Adv. Neural Information Processing Systems 35 (2021), each of which is incorporated by reference in its entirety herein. As another example, a UniRep language model may be used, which model is described in Ma, Eric J., and Arkadij Kummer. “Reimplementing Unirep in JAX.” bioRxiv (2020), which is incorporated by reference in its entirety herein.

In some embodiments, the machine learning model may be formed as an ensemble of multiple machine learning models. For example, the machine learning model may include an ensemble of neural networks. In some embodiments, implementing an ensemble of machine learning models includes training each of multiple (e.g., two or more, three or more, etc.) machine learning models on different training datasets, predicting thermostability using each of the trained machine learning models, and averaging their predictions. In some embodiments, the multiple machine learning models may be combined using boosting.

Regardless of the specific type of machine learning model 108 used as part of the technique 100, the machine learning model outputs an indication of thermo stability indication 110. In some embodiments, that indication may be a temperature or a range of temperatures, at which the scFv is thermostable. For example, the indication may be the temperature that corresponds to the half maximal binding of the scFv (which may be referred to herein as the “TS50” temperature) or a range of temperatures that includes the TS50 temperature. As another example, the indication may be the thermal melting temperature (which may be referred to herein as the “Tₘ” temperature) or a range of temperature that includes the Tₘ temperature. The Tₘ temperature may correspond to the temperature at which the concentration of an scFv in its folded state equals the concentration of the scFv in its unfolded state.

In some embodiments, the machine learning model 108 is configured to output a probability that an scFv is thermostable for each of multiple temperature ranges (e.g., under 50° C., 50-60° C., 60-70° C., over 70° C.). The ranges may be closed (e.g., 50-60° C.) or open (e.g., under 50° C. or over 70° C.). As a nonlimiting example, the machine learning model 108 may output a first probability that the scFv is thermostable in a first temperature range and a second probability that the scFv is thermostable in a second temperature range.

The technique illustrated in FIG. 1A may be used to computationally screen a set of scFvs to identify a subset of the scFvs to produce. For example, the set of scFvs may be screened on the basis of the thermostability indications generated by the technique 100 for the scFvs in the set. As one example, when the thermostability indication is an indication of a temperature (e.g., a TS50 temperature, a Tm temperature), the scFvs having the predicted temperature exceeding a specified threshold (e.g., greater than 50° degrees Celsius (C), greater than 55° C., greater than 60° C.) may be selected for subsequent production.

In some embodiments, the thermostability indication 110 is used to identify scFvs for subsequent production. For example, scFvs having a predicted thermostability indication that meets one or more criteria may be identified for subsequent production. Techniques for screening scFvs for production are described herein including at least with respect to FIG. 2A.

FIG. 1B is a block diagram of an example system 150 for predicting thermostability of scFvs and computationally screening scFvs based on such predictions, in accordance with some embodiments of the technology described herein. System 150 includes computing device(s) 120 that is configured to have software 130 execute thereon to perform various functions in connection with predicting thermostability of an scFv and computationally screening the scFvs based on such predictions.

In the embodiment shown in FIG. 1B, computing device(s) 120 includes software 130 configured to perform various functions with respect to scFv data (e.g., scFv data 104).

The computing device(s) 120 can be one or multiple computing devices of any suitable type. For example, the computing device(s) 120 may be a portable computing device (e.g., laptop, a smartphone) or a fixed computing device (e.g., a desktop computer, a server). When computing device(s) 120 includes multiple computing devices, the device(s) may be physically co-located (e.g., in a single room) or distributed across multiple physical locations. In some embodiments, the computing device(s) 120 may be part of a cloud computing infrastructure.

In some embodiments, the computing device(s) 120 may be operated by one or more user(s) 172 such as one or more researchers and/or other individual(s). For example, the user(s) 172 may provide scFv sequence 102 and/or scFv data 104 as input to the computing device(s) 120 (e.g., by uploading one or more files), and/or may provide user input specifying processing or other methods to be performed on the scFv data.

As shown in FIG. 1B, software 130 includes a plurality of modules. Each module may include processor-executable instructions that, when executed by at least one computer hardware processor, cause the at least one computer hardware processor to perform the function(s) of that module. Such modules are sometimes referred to herein as “software modules.” The modules shown in FIG. 1B, includes processor-executable instructions that, when executed by a computing device, cause the computing device to perform one or more processes, such as the processes described herein including at least with respect to FIGS. 2A-2B, 4A, and 5A-5B. It should be appreciated that the modules shown in FIG. 1B are illustrative and that, in other embodiments, software 130 may be implemented using one or more other software modules, in addition to or instead of, the modules shown in FIG. 1B. In other words, the software 130 may be organized internally differently from how illustrated in FIG. 1B.

As shown in FIG. 1B, software 130 includes multiple software modules for processing scFv data, such as a protein structure prediction module 160, a molecular modeling module 162, a feature generation module 164, a thermostability prediction module 166, and an scFv screening module 168. In the embodiment of FIG. 1B, the software 130 additionally includes a user interface module 170 for obtaining user input.

In some embodiments, the protein structure prediction module 160 obtains scFv data (e.g., scFv data 104) from the scFv data store 146 and/or the user(s) 172 (e.g., by the user uploading the scFv data). In some embodiments, the obtained scFv data includes sequence data (e.g., sequence data 104a) for the scFv.

In some embodiments, the protein structure prediction module 160 may be configured to predict the 3D structure of the scFv using sequence data. For example, the protein structure prediction module 160 may be configured to generate structure data (e.g., structure data 104b) for an scFv from the sequence for that scFv. To this end, the protein structure prediction module 160 may use protein structure prediction software such as, for example, DeepAb software, SAbPred software, or AlphaFold software. Techniques for obtaining information indicative of the 3D structure of an scFv using structure prediction software are described herein including at least with respect to act 252 of process 250, shown in FIG. 2B.

In some embodiments, the molecular modeling module 162 obtains scFv data (e.g., scFv data 104) from the scFv data store 146, the user(s) 172 (e.g., by the user uploading the scFv data), and/or the protein structure prediction module 160. In some embodiments, the obtained scFv data includes sequence data (e.g., sequence data 104a) and/or structure data (e.g., structure data 104b) for the scFv.

In some embodiments, the molecular modeling module 162 is configured to determine interaction energy metrics for residues of an scFv sequence (e.g., scFv sequence 102) in its 3D conformation. For example, the molecular modeling module 162 may be configured to generate interaction energy metrics (e.g., which may be part of energy data 104c) for an scFv. To this end, the molecular modeling module 162 may use molecular modeling software such as, for example, Rosetta software, Schrodinger BioLuminate® or Chemical Computing Group's Molecular Operating Environment (MOE) software may be used. Techniques for determining interaction energy metrics using molecular modeling software are described herein including at least with respect to act 254 of process 250 shown in FIG. 2B.

In some embodiments, the feature generation module 164 obtains scFv data (e.g., scFv data 104) from the scFv data store 146, user(s) 172 (e.g., by the user uploading the scFv data), the molecular modeling module 162, and/or the protein structure prediction module 160 and uses the obtained scFv data to generate sets of features for respective scFvs. For example, the feature generation module 164 may generate a set of features for an scFv having scFv sequence 102.

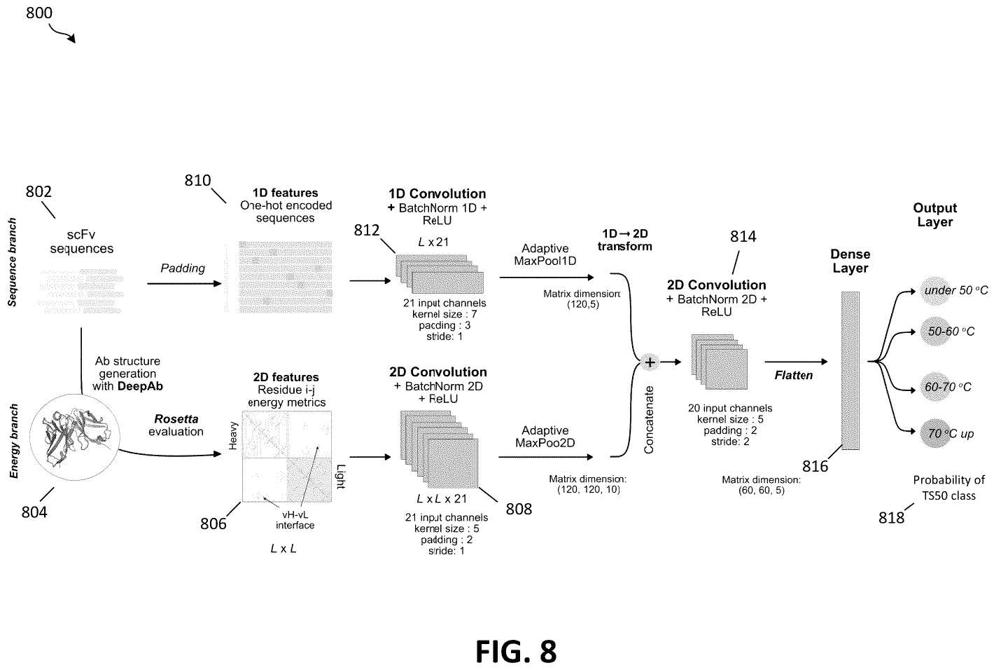

In some embodiments, the feature generation module 164 generates a set of features by including at least some of the obtained data (e.g., scFv data 104) in the set of features. For example, the feature generation module 164 may generate the set of features to include interaction energy metrics for an scFv. For example, feature generation module 164 may generate the set of features to include, for each particular energy metric of the interaction energy metrics, a two-dimensional (2D) matrix that that stores at its (i,j)th location the value of the particular energy metric between the ith and jth residues of the scFv. The 2D matrices so generated may be provided as input to the trained neural network model. For example, as shown in the example FIG. 8, if there are 20 interaction energy metrics, then 20 such matrices may be generated and may be provided as input to a neural network model via 20 input channels. Additionally, or alternatively, the feature generation module 164 may generate a set of features including an encoded sequence for an scFv. For example, the sequence may be one-hot encoded. However, it should be appreciated that the feature generation module 164 may include additional or alternative features in the set of features, as aspects of the technology described herein are not limited in this respect. Techniques for generating a set of features for an scFv are described herein including at least with respect to act 256 of process 250 shown in FIG. 2B.

In some embodiments, the thermostability prediction module 166 obtains one or more sets of features from the feature generation module 164, obtains a trained machine learning model from the machine learning models data store 152 (which may be a data store of any suitable type), and processes the obtained set(s) of features using the obtained machine learning model to obtain thermostability indications for one or more scFvs. For example, the thermostability prediction module 166 may process the set of features generated for the scFv having sequence 102 using the trained machine learning model 108 to obtain a thermostability indication 110 for the scFv. Techniques for predicting thermostability of an scFv using machine learning are described herein including at least with respect to FIG. 2B.

In some embodiments, the scFv screening module 168 is used for computationally screening a set of scFvs in order to identify a subset for subsequent production. To this end, scFv screening module 168 obtains determined thermostability indications from the thermostability prediction module 166 (e.g., by invoking the module 166 to determine thermostability indications for the scFvs in the set) and identifies the scFvs for subsequent production using the thermostability indications. For example, in some embodiments, the scFv screening module 168 compares a thermostability indication to one or more criteria to determine whether the thermostability predictions satisfies the one or more criteria. If the thermostability indication satisfies the criteria, then the scFv screening module 168 may identify the scFv, for which the thermostability was determined, for subsequent production. For example, the thermostability indications may indicate for each scFv a respective temperature or temperature range at which the scFv is thermostable. That output may be compared to a threshold temperature and those scFv's whose indicated temperature is higher than the threshold may pass the screening step and be selected for subsequent production.

In some embodiments, the scFv screening module 168 may perform computational screening based on user input, for example user input provided by user(s) 172 via user interface module 170. The user input may specify one or more criteria for passing scFvs through the screen (e.g., the threshold temperature). Additionally, or alternatively, the user may provide input manually selecting one or more scFvs for subsequent production (e.g., based on their determined thermostabilities or any other factor).

In some embodiments, the protein structure prediction module 160, molecular modeling module 162, and/or feature generation module 164 obtain scFv data via user interface 170 and/or one or more other interface modules (not shown). The data may be provided by a communication network (not shown), such as Internet or any other suitable network, as aspects of the technology described herein are not limited in this respect.

As shown in FIG. 1B, system 150 also includes scFv data store 146 and machine learning model data store 152. In some embodiments, software 130 obtains data from scFv data store 146, machine learning model data store 152, and/or user(s) 172 (e.g., by uploading data). In some embodiments, the software 130 further includes machine learning model training module 154 for training one more machine learning models (e.g., stored in machine learning model data store 152).

In some embodiments, the scFv data is obtained from scFv data store 146. The scFv data store 146 may be of any suitable type (e.g., database system, multi-file, flat file, etc.) and may store scFv data in any suitable way and in any suitable format, as aspects of the technology described herein are not limited in this respect. The scFv data store 146 may be part of or external to computing device(s) 120.

In some embodiments, the scFv data store 146 stores scFv data obtained for an scFv, as described herein including at least with respect to FIG. 1A. In some embodiments, the stored scFv data may have been previously uploaded by a user (e.g., user(s) 172), and/or from one or more public data stores and/or studies. In some embodiments, a portion of the scFv data may be processed by the protein structure prediction module 160 to generate information indicative of a structure of an scFv. In some embodiments, a portion of the scFv data may be processed by the molecular modeling module 162 to determine interaction energies for pairs of residues for an scFv. In some embodiments, a portion of the scFv data may be processed by the feature generation module 164 to generate sets of features for scFvs to be provided as input to a machine learning model. In some embodiments, a portion of the scFv data may be used to train one or more machine learning models (e.g., with the machine learning model training module 154).

In some embodiments, the thermostability prediction module 166 obtains (either pulls or is provided) the trained machine learning model from the machine learning model data store 152. The machine learning models may be provided via a communication network (not shown), such as Internet or any other suitable network, as aspects of the technology described herein are not limited to any particular communication network.

In some embodiments, the machine learning model data store 152 includes any suitable data store, such as a flat file, a data store, a multi-file, or data storage of any suitable type, as aspects of the technology described herein are not limited to any particular type of data store. The machine learning model data store 152 may be part of software 130 (not shown) or excluded from software 130, as shown in FIG. 1B.

In some embodiments, the machine learning model data store 152 stores one or more machine learning models used to predict thermostability for scFvs. The data store 152 may be of any suitable type (e.g., database system, multi-file, flat file, etc.) and may store trained machine learning models in any suitable way and in any suitable format, as aspects of the technology described herein are not limited in this respect. The data store 152 may be part of or external to computing device(s) 120.

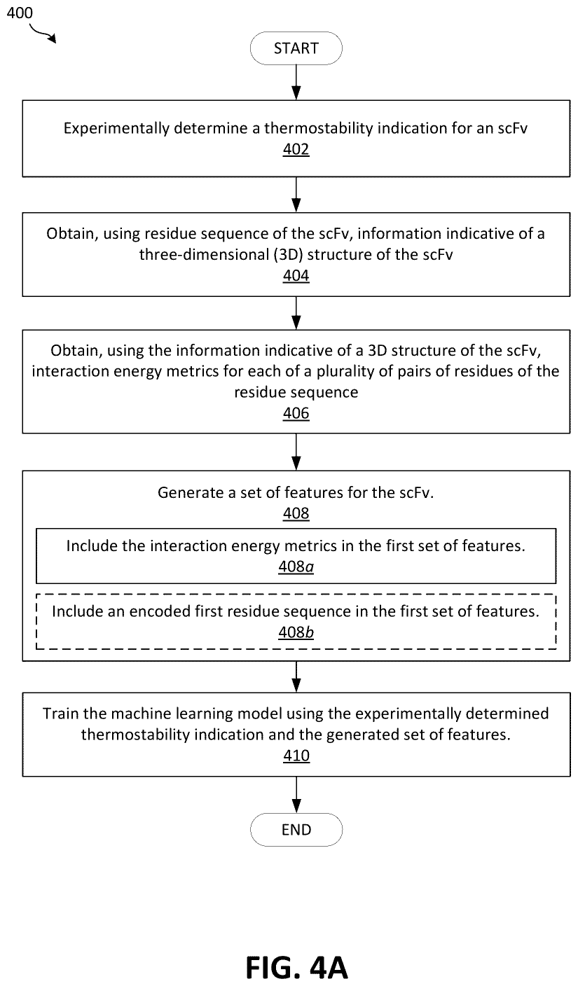

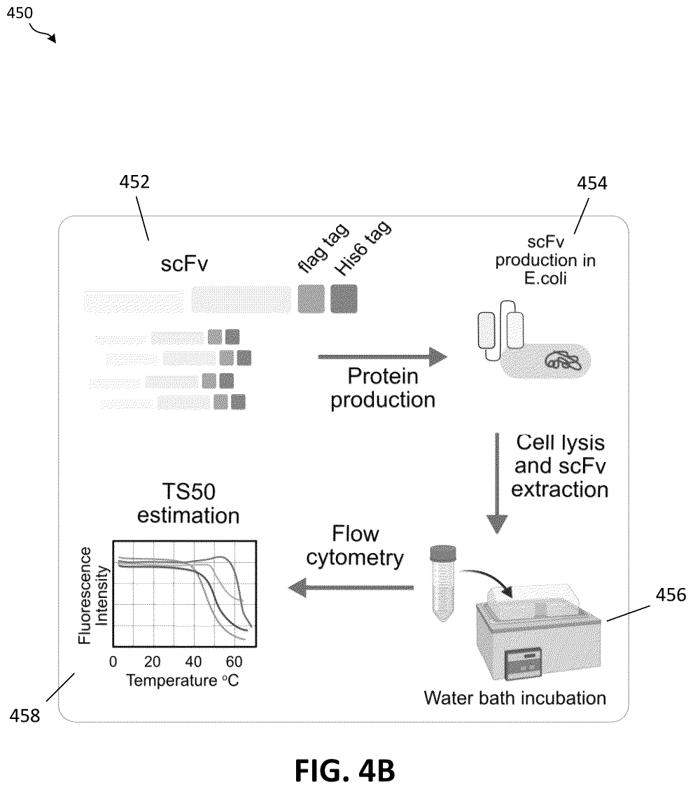

In some embodiments, the machine learning model training module 154, referred to herein as training module 154, may be configured to train the one or more machine learning models to predict thermostability for scFvs. In some embodiments, the training module 154 trains a machine learning model using a training set of scFv data. For example, the training module 154 may obtain training data from the scFv data store 146. In some embodiments, the training module 154 may provide trained machine learning model(s) to the machine learning model data store 152. Techniques for training a machine learning model are described herein including at least with respect to FIG. 4A.

In some embodiments, the predicted thermostability may be output by the thermostability prediction module 166. For example, the predicted thermo stability may be output to user(s) 172 via user interface 170. Additionally, or alternatively, the predicted thermostability may be stored in memory and/or transmitted to one or more other computing devices.

In some embodiments, the scFvs identified for subsequent production may be output by the scFv screening module 168. For example, the identified scFvs may be output to user(s) 172 via user interface 170. Additionally, or alternatively, the identified scFvs may be stored in memory and/or transmitted to one or more other computing devices.

User interface 170 may be a graphical user interface (GUI), a text-based user interface, and/or any other suitable type of interface through which a user may provide input and view information generated by software 130. For example, in some embodiments, the user interface may be a webpage or web application accessible through an Internet browser. In some embodiments, the user interface may be a graphical user interface (GUI) of an app executing on the user's mobile device. In some embodiments, the user interface may include a number of selectable elements through which a user may interact. For example, the user interface may include dropdown lists, checkboxes, text fields, or any other suitable element.

FIGS. 2A-2B are flowcharts depicting illustrative processes for computationally screening a set of scFvs to identify a subset of the set of scFvs to produce, according to some embodiments of the technology described herein.

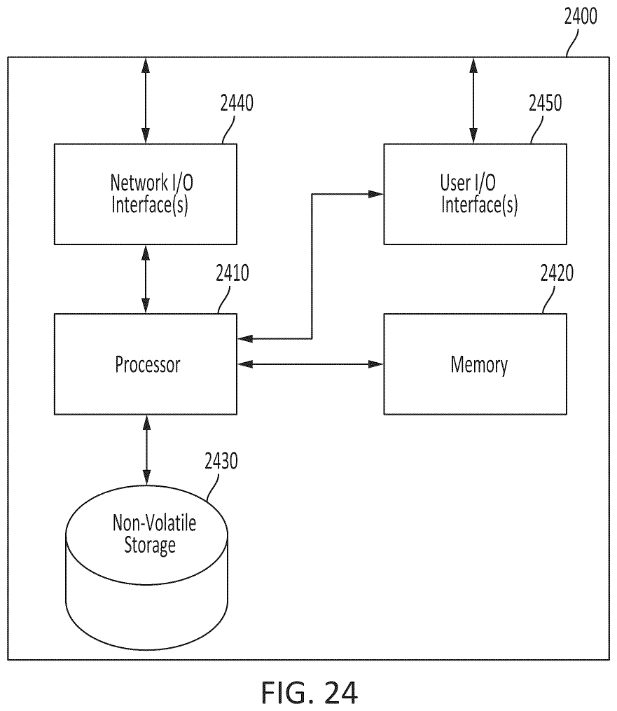

FIG. 2A is a flowchart of an illustrative process 200 for computationally screening a set of scFvs, in accordance with some embodiments of the technology described herein. One or more acts of process 200 may be performed automatically by any suitable computing device(s). For example, the act(s) may be performed by a laptop computer, a desktop computer, one or more servers, in a cloud computing environment, computer system 2400 as described herein within respect to FIG. 24, and/or in any other suitable way. For example, in some embodiments, act 202 may be performed automatically by any suitable computing device(s). As another example, act 204 may be performed automatically by any suitable computing device(s).

Process 200 begins at act 202, where a thermostability indication is determined for each scFv in a set of scFvs using a trained machine learning model. As described above, a thermostability indication may refer to a temperature or a temperature range including at least one temperature at which the scFv is stable.

In some embodiments, determining a thermostability indication of an scFv includes generating a set of features for the scFv and processing the set of features using a trained machine learning model. The output of the machine learning model may be indicative of the thermostability of the scFv (e.g., the thermostability indication). For example, the output may indicate a temperature at which the scFv is thermostable. That temperature may be a TS50 temperature, a Tm temperature or any other type of temperature indicating that the scFv is thermostable. Additionally, or alternatively, the output may indicate a temperature range that includes one or more temperatures at which the scFv is thermostable. Techniques for determining a thermostability indication of a particular scFv using a trained neural network model are described herein including at least with respect to process 250 shown in FIG. 2B.

The set of scFvs may include any suitable number of scFvs. For example, the set of scFvs may include at least 25 scFvs, at least 50 scFvs, at least 75 scFvs, at least 100 scFvs, at least 200 scFvs, at least 300 scFvs, at least 400 scFvs, at least 500 scFvs, at least 600 scFvs, at least 700 scFvs, at least 800 scFvs, at least 900 scFvs, at least 1,000 scFvs, at least 5,000 scFvs, at least 10,000 scFvs, between 100 and 1000 scFvs, between 100 and 10,000 scFvs or any other suitable range within these ranges. Thus, determining the thermostability indications at act 202 may include determining at least 25, at least 50, at least 75, at least 100, at least 200, at least 300, at least 400, at least 500, at least 600, at least 700, at least 800, at least 900, at least 1,000, at least 5,000, at least 10,000, between 100 and 1000, or between 100 and 10,000 thermostability indications.

Process 200 then proceeds to act 204, where a subset of the set of scFvs are identified for subsequent production based on the thermostability indications determined at act 202. In some embodiments, identifying an scFv in the set of scFvs for subsequent production includes identifying an scFv that is thermostable and manufacturable.

In some embodiments, identifying a subset of the scFvs involves determining whether the thermostability indications satisfy one or more criteria. For example, scFvs whose thermostability indications comprise a temperature that exceeds a particular threshold may be identified. As another example, scFvs whose thermostability indications include a particular range of temperatures may be included. When the thermostability indication determined for a particular scFv satisfies one or more criteria, that scFv may be included in the subset of scFvs for subsequent production. If the thermostability indication does not satisfy the one or more criteria, the scFv may be excluded from the subset of scFvs for subsequent production.

As described above, in some embodiments, act 204 may be performed using a computing device (e.g., computing device(s) 120 shown in FIG. 1B). Additionally, or alternatively, act 204 is performed by a user. For example, a user may manually select scFvs for subsequent production based on the thermostability indications output by the trained machine learning model.

In some embodiments, the identified subset includes none, some, or all of the scFvs included in the original set of scFvs. For example, the identified subset may include 0%, less than 10%, less than 25%, less than 50%, less than 75%, less than 90%, or all of the scFvs included in the original set of scFvs.

Process 200 then proceeds to act 206, where at least some of the scFvs included in the identified subset of scFvs are produced. In some embodiments, the scFvs are produced using techniques known in the art.

In some embodiments, producing at least one of the scFvs in the subset of scFvs includes producing one, some, or all of the scFvs in the subset. For example, in some embodiments, at least 10%, at least 25%, at least 50%, at least 75%, at least 90%, or all of the scFvs included in the identified subset are produced at act 206.

In some embodiments, implementing process 200 may include additional or alternative steps that are not shown in FIG. 2A. In some embodiments, process 200 may include only a subset of the acts included in the example flowchart (e.g., act 202 only, acts 202 and 204 only).

FIG. 2B is a flowchart of an illustrative process 250 for determining thermostability for a first scFv, in accordance with some embodiments of the technology described herein. In some embodiments, act 202 of process 200 may implemented using process 250. Process 250 may be performed by any suitable computing device(s) (e.g., computing device(s) 120 shown in FIG. 1B).

Process 250 begins at act 252, where information indicative of a 3D structure of the first scFv is obtained. In some embodiments, this information was previously-obtained for the first scFv. Thus, in some embodiments, obtaining the information indicative of the 3D structure of the first scFv may include accessing the information (e.g., from a memory, over a network, via a file being provided via an appropriate interface, etc.).

In other embodiments, obtaining the information indicative of the 3D structure of the first scFv comprises generating this information. Accordingly, in some embodiments, obtaining the information indicative of the 3D structure of the first scFv includes generating that information by process the residue sequence of the first scFv using protein structure prediction software. The protein structure prediction software may be configured to output the information indicative of the 3D structure of the first scFv. Any suitable protein structure prediction software may be used. For example, DeepAb software may be used, aspects of which are described in Ruffolo, Jeffrey A., Jeremias Sulam, and Jeffrey J. Gray. “Antibody structure prediction using interpretable deep learning.” Patterns 3.2 (2022): 100406, which is incorporated by reference in its entirety herein. As another example, SAbPred software may be used, aspects of which are described in Dunbar, James, et al. “SAbPred: a structure-based antibody prediction server.” Nucleic acids research 44.W1 (2016): W474-W478, which is incorporated by reference in its entirety herein. As yet another example, AlphaFold software may be used, aspects of which are described in Jumper, Johns, et al. “Highly accurate protein structure prediction with AlphaFold.” Nature 596, 583-589 (2021), which is incorporated by reference herein in its entirety.

Process 250 then proceeds to act 254, where interaction energy metrics for each of a plurality of pairs of amino acid residues of the first scFv are obtained. In some embodiments, obtaining the interaction energy metrics includes generating the energy metrics by processing the information indicative of the 3D structure of the first scFv using molecular modeling software. The molecular modeling software may be configured to output the interaction energy metrics for the first scFv. Any molecular modeling software capable of estimating residue interaction energy metrics may be used. For example, the Rosetta molecular modeling software may be used. Rosetta software and techniques used by it to estimate residue interaction energy metrics are described in: Alford, et al. (The Rosetta All-Atom Energy Function for Macromolecular Modeling and Design. 440 J. Chem. Theory Comput. 13, 3031-3048 (2017), Chaudhury, et al. (PyRosetta: a script-based interface for implementing molecular modeling algorithms using Rosetta, Bioinformatics, 26(5), 689-691 (2010)), and Leaver-Fay, et al. (Rosetta3: An object-oriented software suite for the simulation and design of macromolecules, In Methods in Enzymology, 545-574), each of which is incorporated by reference herein in its entirety. As another example, Schrodinger's BioLuminate® and/or Prime software may be used (Schrodinger Suite 2020-2 release, Schrodinger Inc, New York, NY). As yet another example, Chemical Computing Group's Molecular Operating Environment (MOE) software may be used (Molecular Operating Environment (MOE), 2020.09 Chemical Computing Group ULC, 1010 Sherbooke St. West, Suite #910, Montreal, QC, Canada, H3A 2R7, 2022).

In some embodiments, interaction energy metrics are obtained for some or all of the pairs of residues of the residue sequence of the first scFv. For example, interaction energy metrics may be obtained for at least 75%, at least 80%, at least 85%, at least 90%, at least 95%, at least 98%, at least 99%, or all of the pairs of residues of the amino acid sequence of the first scFv.

Any of numerous types of interaction energy metric(s) may be obtained for a pair of residues. For example, the interaction energy metric(s) for a pair of residues may include one or more of Lennard-Jones attraction and/or repulsion energies, and Van der Waals energies (e.g., “fa_atr:—the attractive energy between two atoms on different residues separated by a distance d, “fa_rep”—repulsive energy between two atoms on different residues separated by a distance d, and/or “fa_intra-rep”—repulsive energy between two atoms on the same residue separated by a distance d). As another example, interaction energy metric(s) for a pair of residues may include one or more solvation energies (e.g., “fa_sol”—Gaussian exclusion implicit solvation energy between protein atoms in different residues, “fa_intra_sol”—Gaussian exclusion implicit solvation energy between protein atoms in the same residue; and/or “lk_ball_wtd”—orientation-dependent solvation of polar atoms assuming ideal water geometry). As another example, interaction energy metric(s) for a pair of residues an electrostatics energy (e.g., “fa_elec”—energy of interaction between two nonbonded charges atoms separated by a distance d). As another example, interaction energy metric(s) for a pair of residues may include one or more of hydrogen bond and/or disulfide bridge energies (e.g., “hbond_lr_bb”—energy of long-range hydrogen bonds; “hbond_sr_bb”—energy of short-range hydrogen bonds; “hbond_bb_sc”—energy of backbone-side chain hydrogen bonds; “hbond_sc”—energy of side-chain-side-chain hydrogen bonds; “dslf_fal3”—energy of disulfide bridges). As another example, interaction energy metric(s) for a pair of residues may include one or more backbone statistics (e.g., “rama_prepro”—probability of backbone angles φ and ψ given the amino acid type; “omega”—backbone-dependent penalty for cis φ dihedrals that deviate from 0° and trans ω dihedrals that deviate from 180°; “p_aa_pp”—probability of amino acid identity give backbone φ and Ψ angles; “pro_close”—penalty for an open proline ring and proline ω bonding energy; “yhh_planarity”—sinusoidal penalty for nonplanar tyrosine χ₃ dihedral angle). As another example, interaction energy metric(s) for a pair of residues may include a knowledge-based rotamer energy (e.g., “fa_dun”—probability that a chose rotamer is native-like give the backbone values). Accordingly, interaction energy metrics may include any (some or all) of the foregoing examples of energy metrics. These foregoing examples are described further in Alford, et al. (The Rosetta All-Atom Energy Function for Macromolecular Modeling and Design. 440 J. Chem. Theory Comput. 13, 3031-3048 (2017), which is incorporated by reference herein in its entirety. One or more other energy metrics may be used in addition to or instead of any one or more of the foregoing example energy metrics.

Process 250 then proceeds to act 256, where a first set of features is generated for the first scFv so that the first of features may be provided as input to the trained machine learning model at act 258 to obtain a corresponding output indicative of a first thermostability for the first scFv. In some embodiments, generating the first set of features involves including, in the first set of features, at least some of the data obtained at act 252 and/or act 254 of process 250.

For example, at act 256a, interaction energy metrics are included in the first set of features. In some embodiments, the interaction energy metrics include some or all of the interaction energy metrics obtained at act 254, examples of which are provided herein.

In some embodiments, including the interaction energy metrics in the first set of features at act 256a includes generating one or more matrices of interaction energy metrics. For example, for each particular energy metric, a respective 2D matrix may be generated that has, in its entry in the ith row and jth column, the value of that particular metric for the ith and jth residues. When the interaction energy metrics include multiple metrics, a 2D matrix may be generated for each of them and therefore included as part of the first set of features being generated at act 256a of process 250. The 2D matrices may be therefore provided as inputs (e.g., via different channels as shown in FIG. 8) to the trained machine learning model. Alternatively, a 3D matrix may be generated instead of multiple 2D matrices, as aspects of the technology described herein are not limited in this respect.

Additionally, or alternatively, at act 256b, an encoded first residue sequence is included in the first set of features. In some embodiments, encoding the first residue sequence to obtain the encoded first residue sequence may be performed using any suitable encoding technique, such as one-hot encoding. In some embodiments, the first residue sequence is encoded to form a particular input dimension. For example, the first residue sequence may be encoded to form an input dimension of (V_{H}+V_{L}+3)×21, where V_{H} and V_{L} correspond to the heavy and light chain sequences of the scFv residue sequence, respectively.

Though not shown in FIG. 2B, it should be appreciated that one or more additional or alternative features may be included in the first set of features, as aspects of the technology described herein are not limited in this respect.

Next, process 250 then proceeds to act 258, where the first set of features is provided as input to the trained machine learning model to obtain an output indicative of the thermostability of the first scFv.

In some embodiments, the machine learning model is of any suitable type. For example, the machine learning model may be a neural network, such as a Convolutional Neural Network (CNN). The CNN may have one or more two-dimensional convolutional layer, one or more three-dimensional convolutional layer, and/or a fully connected layer.

As one specific example, the CNN may have the architecture shown in the FIG. 8, which includes one or more max pooling layers (Adaptive MaxPool 1D and Adaptive Max Pool2D layers in FIG. 8), one or more 2-dimensional convolution layers, one or more non-linearity layers (e.g., a rectified linear unit layer “ReLu” in FIG. 8), one or more batch normalization layers, and dense layer. The architecture shown in FIG. 8 shows that both energy metrics and one-hot encoded sequences are provided as input. However, as described herein, energy metrics only or one-hot encoded sequences only may be provided as input, in some embodiments. Thus, in some embodiments, either the top branch or the lower branch or both branches of the architecture shown in FIG. 8 may be used.

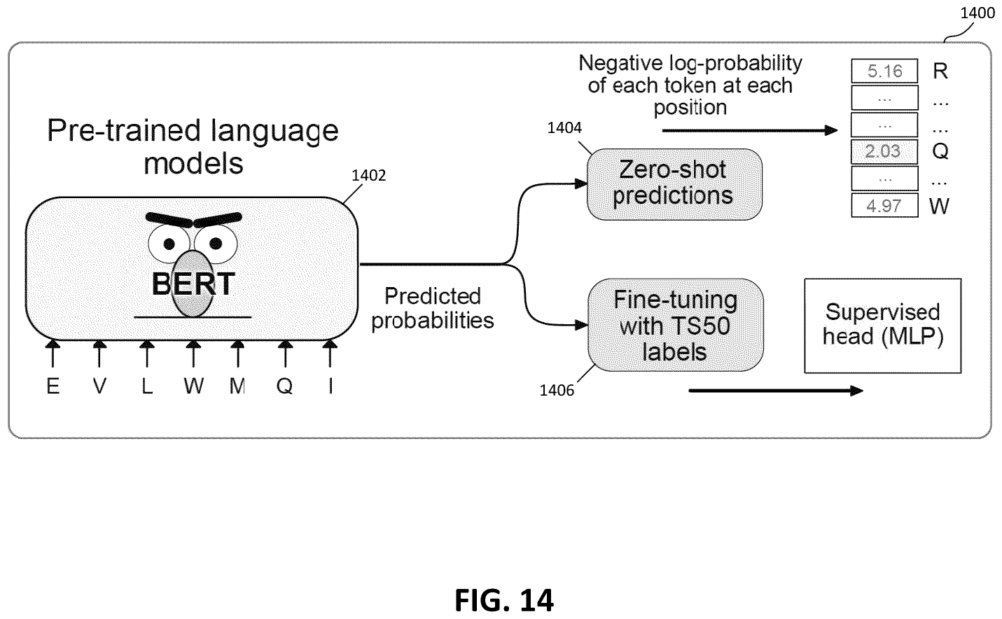

As another example, the machine learning model may be a pre-trained language model (used either to perform zero-shot predictions or tuned by adding a supervised head using transfer learning with a small amount of training data), such as a bidirectional encoder representations from transformers (BERT) model (e.g., an ESM-1b language model or the ESM-1v language model) or a UniRep language model, which are described herein. Such models may be used when the input only includes sequence features and does not include energy features (e.g., as shown in FIG. 14, for example).

In some embodiments, the machine learning model includes multiple machine learning models (e.g., an ensemble of machine learning models). For example, the machine learning model may include an ensemble of neural networks. In some embodiments, implementing an ensemble of machine learning models includes training each of multiple (e.g., two or more, three or more, etc.) machine learning models on different training datasets, predicting thermostability using each of the trained machine learning models, and averaging (e.g., weighted averaging of) their predictions. Outputs of multiple machine learning models may be combined in any other way, as aspects of the technology described herein are not limited in this respect. The ensemble of machine learning models may be obtained, in part, by using any suitable bagging or boosting technique.

In some embodiments, the machine learning model is trained to process the first set of features to obtain the probability that the thermostability of the first scFv belongs to each of multiple classes of thermostabilities. For example, this may include determining the probability that the first scFv is thermostable in each of a plurality of temperature ranges. As a nonlimiting example, classes of thermostabilities may include the following four classes: under-50° C., 50° C.-60° C., 60° C.-70° C., and 70° C.-up.

In some embodiments, based on the determined probabilities, the machine learning model is configured to predict a class of the multiple classes associated with the highest probability. For example, this may include identifying a temperature range of multiple temperature ranges associated with the highest probability. The identified temperature range includes one or more temperatures at which the first scFv is thermostable.

Additionally, or alternatively, based on the determined probabilities, the machine learning model may be configured to determine a temperature at which the first scFv is thermostable. For example, the temperature may be the TS50 temperature. As another example, the temperature may correspond to the thermal melting temperature (Tm.) In some embodiments, determining the temperature at which the first scFv is thermostable includes determining, as the temperature, a weighted linear combination of the temperature range means weighted by the probabilities determined using the machine learning model.

At act 260, process 250 includes determining whether there is another scFv in the set of scFvs for which thermostability can be determined. When it is determined, at act 260, that there is another scFv for which thermostability is to be determined, acts 252-258 are repeated for the other scFv. For example, for a second scFv, this would include determining a second set of features and providing the second set of features as input to the trained machine learning model to determine a thermostability indication for the second scFv.

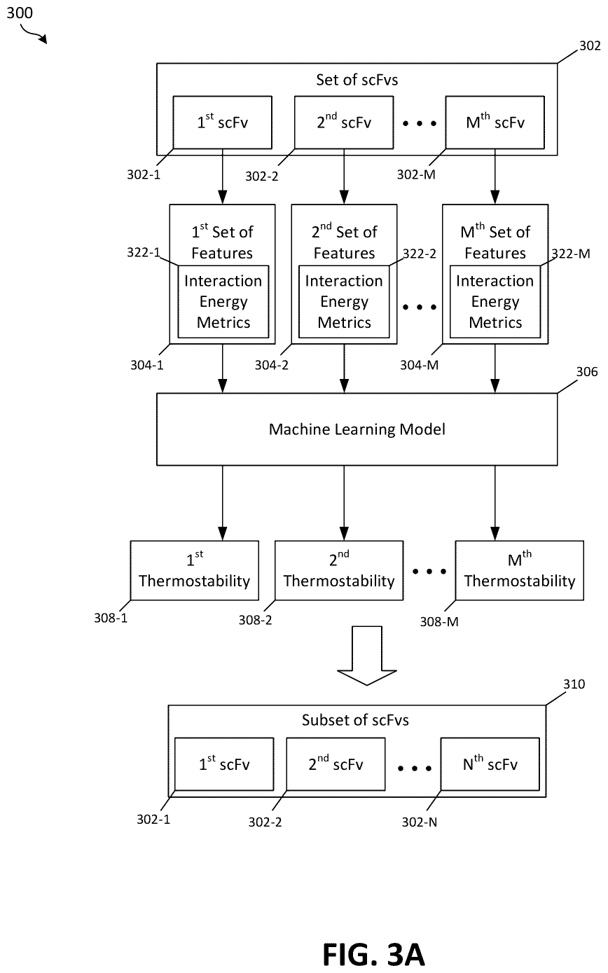

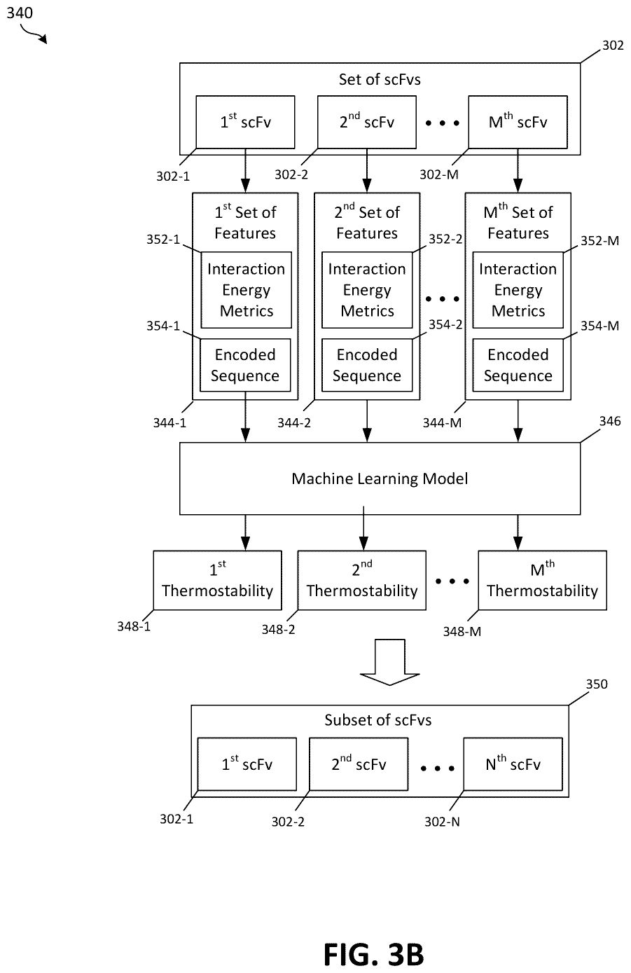

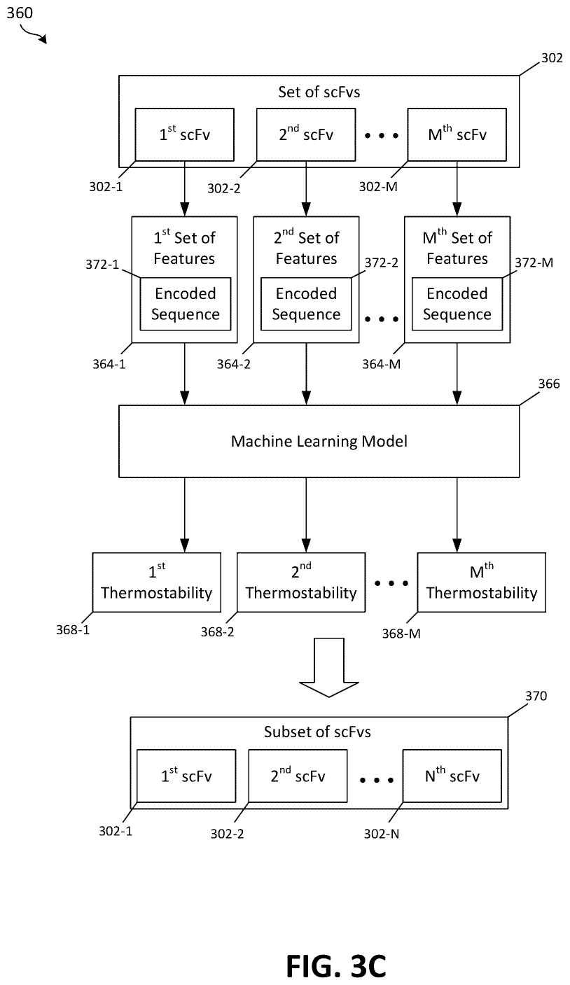

FIG. 3A is a diagram of an illustrative technique for computationally screening a set of scFvs using a trained machine learning model that is trained to generate a thermostability indication for an scFv from input containing interaction energy metrics of pairs of residues in the scFv, in accordance with some embodiments of the technology described herein.

As shown in the embodiment of FIG. 3A, example technique 300 begins with a set of scFvs 302. In some embodiments, the set of scFvs 302 are candidates for production. For example, scFvs included in the set 302 may have one or more desirable characteristics (e.g., affinity, specificity, etc.). The set of scFvs may have any suitable size M, examples of which are provided herein including with reference to FIG. 2A. In the example of FIG. 3A, the set of scFvs 302 includes a first scFv 302-1, a second scFv 302-2, and an Mth scFv 302-M.

Each scFv in the set 302 has a respective residue sequence. For example, the first scFv 302-1 has a first residue sequence, the second scFv 302-2 has a second residue sequence, and the Mth scFv 302-M has an Mth residue sequence. In some embodiments, the first, second, and Mth residue sequences are different from one another. For example, the sequences may differ from one another by one or more residues.

As shown in FIG. 3A, example technique 300 involves generating a set of features for each scFv included in the set of scFvs 302. This includes, for example, generating a first set of features 304-1 for the first scFv 302-1, generating a second set of features 304-2 for the second scFv 302-2 and generating an Mth set of features 304-M for the Mth scFv 302-M. Techniques for generating a set of features are described herein including at least with respect to act 256 of process 250 shown in FIG. 2B.

In some embodiments, the set of features generated for an scFv includes interaction energy metrics for each of multiple pairs of residues in the scFv. For example, as shown in FIG. 3A, the first set of features 304-1 includes interaction energy metrics 322-1 for each of multiple pairs of residues of the first residue sequence, the second set of features 304-2 includes interaction energy metrics 322-2 for each of multiple pairs of residues of the second residue sequence, and the Mth set of features 304-M includes interaction energy metrics 322-M for each of multiple pairs of residues of the Mth residue sequence. Techniques for obtaining interaction energy metrics for pairs of residues are described herein including at least with respect to FIG. 2B.

In the embodiment shown in FIG. 3A, after generating a set of features for each scFv in the set of scFvs 302, the generated sets of features 304-1, 304-2, . . . 304-M are provided as inputs to the trained machine learning model 306.

In some embodiments, the machine learning model 306 is trained to predict a thermostability indication of an scFv based on the set of features provided as input to the machine learning model 306. For example, the first set of features 304-1 may be provided to the machine learning model 306 to obtain an output 308-1 indicative of the thermostability of the first scFv 302-1. The second set of features 304-2 may be provided as input to the machine learning model 306 to obtain an output 308-2 indicative of the thermostability of the second scFv 302-2. The Mth set of features 304-M may be provided as input to the machine learning model 306 to obtain an output 308-M indicative the thermostability of the Mth scFv 302-M. Techniques for using a machine learning model for predicting thermostability of scFvs are described herein including at least with respect to FIG. 2B.

In some embodiments, the example technique 300 includes, identifying, based on the determined thermostability indications (e.g., first thermostability 308-1, second thermostability 308-2, and Mth thermostability 308-M) a subset 310 of the set of scFvs 302. In some embodiments, identifying scFvs to be included in the subset 310 includes identifying scFvs having thermostabilities that meet one or more criteria. This may include, for example, comparing the thermostabilities to a threshold temperature and identifying those scFvs having a thermostability that exceeds the threshold. The identified scFvs may have thermostabilities that make them suitable for production. Techniques for identifying scFvs for subsequent production are described herein including at least with respect to act 204 of process 200 shown in FIG. 2A.

In some embodiments, a subset of scFvs includes one or more of scFvs included in the original set 302 of scFvs. As shown in the embodiment of FIG. 3A, the identified subset 310 includes N scFvs. For example, the subset 310 includes the first scFv 302-1, the second scFv 302-2, and the Nth scFv 302-N. In some embodiments, N is less than 10%, less than 20%, less than 30%, less than 40%, less than 50%, less than 60%, less than 70%, less than 80%, or less than 90%, or less than 100% of M. In some embodiments, N is equal to M.

In some embodiments, at least one of the scFvs included in a subset of scFvs are subsequently produced. For example, one, some, or all of the scFvs included in subset 310 may be subsequently produced.

FIG. 3B is a diagram of an illustrative technique for computationally screening a set of scFvs using a trained machine learning model that is trained to generate a thermostability indication for an scFv from input containing interaction energy metrics of pairs of residues in the scFv and features representing the sequence of the scFv, in accordance with some embodiments of the technology described herein.

As shown in the embodiment of FIG. 3B, example technique 340 begins with a set of scFvs 302. An example set of scFvs is described herein including at least with respect to FIG. 3A.

In some embodiments, the example technique 340 includes generating sets of features for each scFv included in the set of scFvs 302. This includes, for example, generating a first set of features 344-1 for the first scFv 302-1, generating a second set of features 344-2 for the second scFv 302-2 and generating an Mth set of features 344-M for the Mth scFv 302-M. Techniques for generating a set of features are described herein including at least with respect to act 256 of process 250 shown in FIG. 2B.

In some embodiments, a set of features includes interaction energy metrics for each of multiple pairs of residues for a respective scFv. For example, as shown in FIG. 3B, the first set of features 344-1 includes interaction energy metrics 352-1 for each of multiple pairs of residues of the first residue sequence, the second set of features 344-2 includes interaction energy metrics 352-2 for each of multiple pairs of residues of the second residue sequence, and the Mth set of features 344-M includes interaction energy metrics 352-M for each of multiple pairs of residues of the Mth residue sequence. Techniques for obtaining interaction energy metrics for pairs of residues are described herein including at least with respect to FIG. 2B.

Additionally, in the embodiment shown in FIG. 3B, a set of features includes an encoded residue sequence for the respective scFv. For example, the first set of features 344-1 includes encoded sequence 354-1 for the first scFv 302-1, the second set of features 344-2 includes encoded sequence 354-2 for the second scFv 302-2, and the Mth set of features 344-M includes encoded sequence 354-M for the Mth scFv 302-M. Techniques for obtaining encoded residue sequences are described herein including at least with respect to FIG. 2B.

In the embodiment shown in FIG. 3B, after generating a set of features for each scFv in the set of scFvs 302, example technique 340 includes providing the generated sets of features as inputs to the trained machine learning model 346.

In some embodiments, the machine learning model 346 is trained to predict a thermostability indication of an scFv based on the set of features provided as input to the machine learning model 346. For example, the first set of features 344-1 may be provided to the machine learning model 346 to obtain an output 348-1 indicative of the thermostability of the first scFv 302-1. The second set of features 344-2 may be provided as input to the machine learning model 346 to obtain an output 348-2 indicative of the thermostability of the second scFv 302-2. The Mth set of features 344-M may be provided as input to the machine learning model 346 to obtain an output 348-M indicative the thermostability of the Mth scFv 302-M. Techniques for using a machine learning model for predicting thermostability of scFvs are described herein including at least with respect to FIG. 2B.

In some embodiments, the example technique 340 includes, identifying, based on the determined thermostability indications (e.g., first thermostability 348-1, second thermostability 348-2, and Mth thermostability 348-M) a subset 350 of the set of scFvs 302. Example techniques for identifying a subset of scFvs are described herein including at least with respect to FIG. 3A.

FIG. 3C is a diagram of an illustrative technique for computationally screening a set of scFvs using a trained machine learning model that is trained to generate a thermostability indication for an scFv from input containing features representing the sequence of the scFv, in accordance with some embodiments of the technology described herein.

As shown in the embodiment of FIG. 3C, example technique 360 begins with a set of scFvs 302. An example set of scFvs is described herein including at least with respect to FIG. 3A.

In some embodiments, the example technique 360 includes generating sets of features for each scFv included in the set of scFvs 302. This includes, for example, generating a first set of features 364-1 for the first scFv 302-1, generating a second set of features 364-2 for the second scFv 302-2 and generating an Mth set of features 364-M for the Mth scFv 302-M. Techniques for generating a set of features are described herein including at least with respect to act 256 of process 250 shown in FIG. 2B.

In some embodiments, a set of features includes an encoded residue sequence for the respective scFv. For example, the first set of features 364-1 includes encoded sequence 372-1 for the first scFv 302-1, the second set of features 364-2 includes encoded sequence 372-2 for the second scFv 302-2, and the Mth set of features 364-M includes encoded sequence 372-M for the Mth scFv 302-M. Techniques for obtaining encoded residue sequences are described herein including at least with respect to FIG. 2B.

In the embodiment shown in FIG. 3C, after generating a set of features for each scFv in the set of scFvs 302, example technique 360 includes providing the generated sets of features as inputs to the trained machine learning model 366.

In some embodiments, the machine learning model 366 is trained to predict a thermostability indication of an scFv based on the set of features provided as input to the machine learning model 366. For example, the first set of features 364-1 may be provided to the machine learning model 366 to obtain an output 368-1 indicative of the thermostability of the first scFv 302-1. The second set of features 364-2 may be provided as input to the machine learning model 366 to obtain an output 368-2 indicative of the thermostability of the second scFv 302-2. The Mth set of features 364-M may be provided as input to the machine learning model 366 to obtain an output 368-M indicative the thermostability of the Mth scFv 302-M. Techniques for using a machine learning model for predicting thermostability of scFvs are described herein including at least with respect to FIG. 2B.

In some embodiments, the example technique 360 includes, identifying, based on the determined thermostability indications (e.g., first thermostability 368-1, second thermostability 368-2, and Mth thermostability 368-M) a subset 370 of the set of scFvs 302. Example techniques for identifying a subset of scFvs are described herein including at least with respect to FIG. 3A.

FIG. 4A is a flowchart of an illustrative process 400 for training a machine learning model to generate a thermostability indication for an scFv, in accordance with some embodiments of the technology described herein. The process 400 may be performed by any suitable computing device(s). For example, the processes may be performed by a laptop computer, a desktop computer, one or more servers, in a cloud computing environment, computer system 2400 as described herein within respect to FIG. 24, or in any other suitable way. In some embodiments, a software module, such as machine learning model training module 154 as described herein with respect to FIG. 1B, includes processor-executable instructions that, when executed by a computing device, cause the computing device to perform process 400.

Process 400 begins at act 402, where a thermostability indication is experimentally determined for an scFv. In some embodiments, experimentally determining a thermostability indication includes producing the scFv and analyzing the produced scFv to determine the experimental thermostability indications. This may include, for example, experimentally determining, as the thermostability indication, the temperature (TS50) corresponding to half maximal binding and/or the thermal melting (Tm) temperature for an scFv. Techniques for producing scFvs and experimentally determining thermostability indications are described herein including at least with respect to FIG. 4B.

Process 400 then proceeds to act 404, where information indicative of the 3D structure of the scFv is obtained using the residue sequence of the scFv. Techniques for obtaining information indicative of a 3D structure of an scFv are described herein including at least with respect to act 252 of process 250 shown in FIG. 2B.

Process 400 then proceeds to act 406, where interaction energy metrics for pairs of the residue sequence of the scFv are obtained using the information of the 3D structure of the scFv. Techniques for obtaining interaction energy metrics are described herein including at least with respect to act 254 of process 250 shown in FIG. 2B.

When the machine learning model takes in features of a particular type, then process 400 includes generating features of that type and using them for training. The types of features may include, for example, energy features only, sequence features only, or a combination of energy and sequence features. For example, at act 408, process 400 includes generating a set of features for an scFv. Generating the set of features may include, at act 408a, including, in the set of features, interaction energy metrics for pairs of residues of the scFv residue sequence. Additionally, or alternatively, generating the set of features may include, at act 408b, including, in the set of features, an encoded residue sequence for the scFv. Techniques for generating a set of features for an scFv are described herein including at least with respect to act 256 of process 250, shown in FIG. 2B.

In some embodiments, when the machine learning model takes in sequence features, training the machine learning model may include providing the sequence features to the machine learning model in a particular order. For example, as described herein, the sequence features may include encoded residue sequences. In the case of an scFv, an encoded scFv sequence may be provided to the machine learning model as an encoded VH sequence followed by an encoded VL sequence (“VH-VL”), or as an encoded VL sequence followed by an encoded VH sequence (“VL-VH”).

In some embodiments, the machine learning model may be trained using training data that includes sequences all in the same order. For example, each sequence in the training data may consist of an encoded VH sequence followed by an encoded VL sequence. As another example, each sequence in the training data may consist of an encoded VL sequence followed by an encoded VH sequence.

If, during training, all of the sequence data is provided to the machine learning model in the same order (e.g., either all VH-VL or all VL-VH), then new data may also be provided in that same order. If new sequence data is provided in a different order, then the machine learning model may not perform as well in processing the new data, since it was not trained on data provided in that order. For example, if the machine learning model was trained on scFV sequences specified only in the VH-VL order, then it may not perform as well in processing new sequences that are provided in the VL-VH order. Indeed, with respect to scFvs, a VH-VL molecule can be physically different from an VL-VH molecule. By training the machine learning model on scFv sequences specified in only one order, the machine learning model may not have an opportunity to learn about the underlying physical differences of scFv molecules specified in the other order. As a result, the machine learning model may not perform as well in processing scFv sequences provided in the other order. In contrast, for an antibody, because the VH and VL can be separate chains, whether the model sees VH-VL or VL-VH can be purely a matter of notation, the underlying physical entity can be the same, and therefore performance differences can be due to the input order alone. As a result, performance differences due to input order may not be as substantial when processing antibodies as when processing scFVs.

In some embodiments, training the machine learning model includes providing to the machine learning model, sequence data in both orders. For example, sequence data may be provided in both the order of an encoded VL sequence following by an encoded VH sequence, as well as in the order of an encoded VH sequence following by an encoded VL sequence. If, during training, the sequence data is provided to the machine learning model in both orders (e.g., both VH-VL and VL-VH), then new data may be provided to the machine learning model in both orders. Because the machine learning model was trained using sequence data provided in both orders, its performance may be consistent regardless of the order in which new sequence data is provided.

Process 400 then proceeds to act 410, where a machine learning model is trained using the experimentally determined thermostability indication determined at act 402 and the set of features generated at act 408.

In some embodiments, training the machine learning model at act 410 includes estimating parameters of the machine learning model from training data. In some embodiments, the estimation may be done iteratively (e.g., using iterative gradient descent techniques). In some embodiments, the estimation may be done using optimization software to adjust the parameters. For example, in some embodiments, the ADAM optimizer may be used, aspects of which are described in Kingma, Diederik P., and Jimmy Ba. “Adam: A Method for Stochastic Optimization.” In Proceedings of the 3rd International Conference on Learning Representations, ICLR (2015), which is incorporated by reference in its entirety herein. In some embodiments, estimating parameters includes estimating weights of the connections.

In some embodiments, any suitable hyperparameters may be used during training at act 410. The hyperparameters may be set manually or using any other suitable method. Nonlimiting examples of hyperparameters include number of layers, batch size, number of filters, kernel size, epoch, pooling size, and learning rate. However, it should be appreciated that any other suitable hyperparameters may be used during training at act 410.

FIG. 4B is a diagram of an example technique 450 for experimentally generating data used to train a model to predict thermostability of an scFv 452, in accordance with some embodiments of the technology described herein. As shown, scFvs are produced at 454, in an E. coli culture, for example. Cells are then lysed (e.g., using freeze/thaw cycles), and the scFvs are extracted and incubated at 456. After incubation, the lysates may be incubated with target transfected cells (e.g., CHO-cells) and bound scFvs may be detected and analyzed by flow cytometry or any other suitable technique. In some embodiments, the results of the analysis are used to estimate thermostability at 458. For example, this may include estimating a TS50 value and/or a Tₘ value for the scFv. Example techniques for producing scFvs and experimentally estimating thermostability of the scFvs are described herein including at least with respect to the sections “Generation of scFvs,” “TS50 Screening Assay,” and “nanoDSFT Method.”

The techniques described herein are not limited to being applied to single-chain variable fragments and may also be applied to other constructs described herein. For example, as described above, the techniques described herein can be applied to any types of antibodies. An antibody sequence may be used to predict structure, which in turn may be used to calculate energy metrics. Then the energy metrics (and, optionally, an encoding of the input sequence) may be provided as input to the trained machine learning model to obtain a thermostability indication for the antibody.

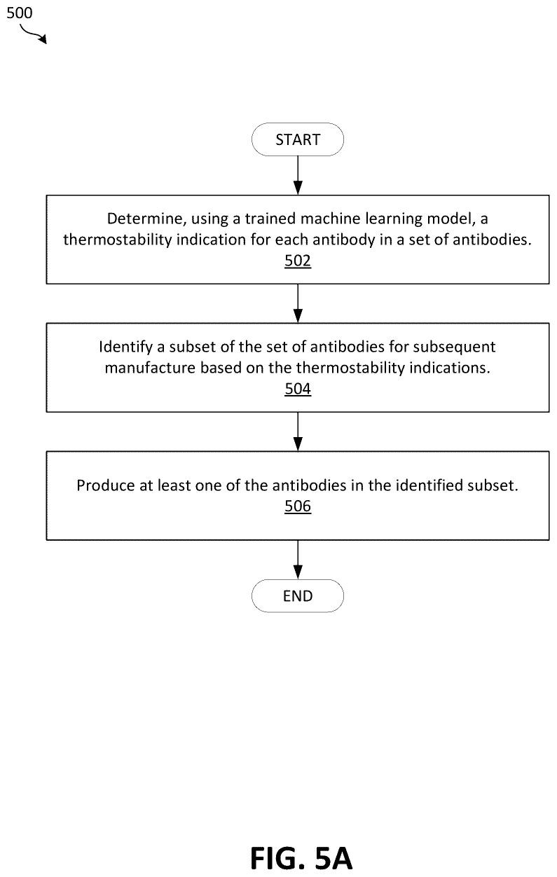

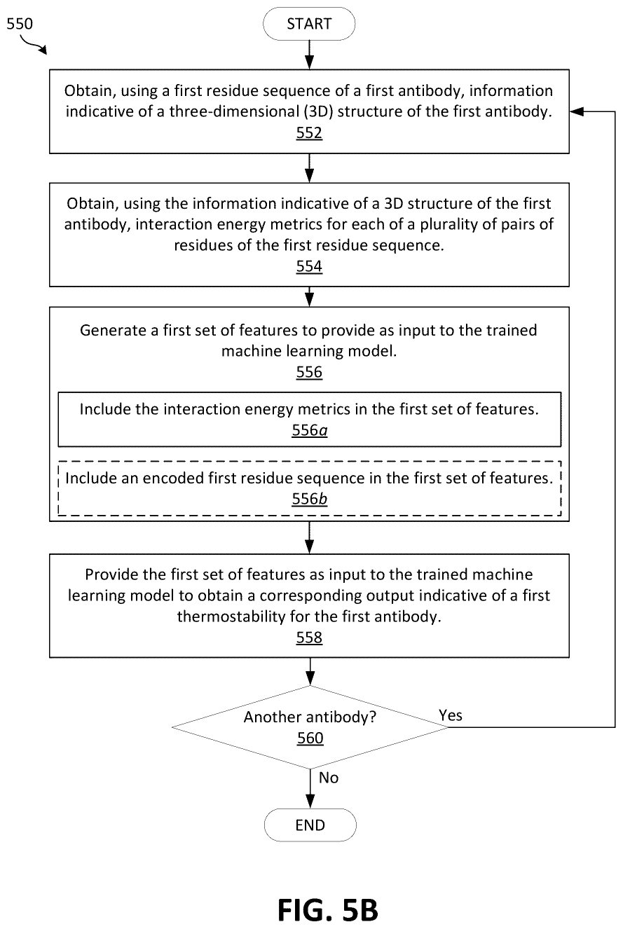

FIG. 5A is a flowchart of an illustrative process 500 for computationally screening a set of antibodies, in accordance with some embodiments of the technology described herein. One or more acts of process 500 may be performed automatically by any suitable computing device(s). For example, the act(s) may be performed by a laptop computer, a desktop computer, one or more servers, in a cloud computing environment, computer system 2400 as described herein within respect to FIG. 24, and/or in any other suitable way. For example, in some embodiments, act 502 may be performed automatically by any suitable computing device(s). As another example, act 504 may be performed automatically by any suitable computing device(s).

Process 500 begins at act 502, where a thermostability indication is determined for each antibody in a set of antibodies using a trained machine learning model. As described above, a thermostability indication may refer to a temperature or a temperature range including at least one temperature at which the antibody is stable.

In some embodiments, determining a thermostability indication of an antibody includes generating a set of features for the antibody and processing the set of features using a trained machine learning model. The output of the machine learning model may be indicative of the thermostability of the antibody (e.g., the thermostability indication). For example, the output may indicate a temperature at which the antibody is thermostable. That temperature may be a TS50 temperature, a Tm temperature or any other type of temperature indicating that the antibody is thermostable. Additionally, or alternatively, the output may indicate a temperature range that includes one or more temperatures at which the antibody is thermostable. Techniques for determining a thermostability indication of particular antibody using a trained neural network model are described herein including at least with respect to process 550 shown in FIG. 5B.

The set of antibodies may include any suitable number of antibodies. For example, the set of antibodies may include at least 25 antibodies, at least 50 antibodies, at least 75 antibodies, at least 100 antibodies, at least 200 antibodies, at least 300 antibodies, at least 400 antibodies, at least 500 antibodies, at least 600 antibodies, at least 700 antibodies, at least 800 antibodies, at least 900 antibodies, at least 1,000 antibodies, at least 5,000 antibodies, at least 10,000 antibodies, between 100 and 1000 antibodies, between 100 and 10,000 antibodies or any other suitable range within these ranges. Thus, determining the thermostability indications at act 502 may include determining at least 25, at least 50, at least 75, at least 100, at least 200, at least 300, at least 400, at least 500, at least 600, at least 700, at least 800, at least 900, at least 1,000, at least 5,000, at least 10,000, between 100 and 1000, or between 100 and 10,000 thermostability indications.

Process 500 then proceeds to act 504, where a subset of the set of antibodies are identified for subsequent production based on the thermostability indications determined at act 502. In some embodiments, identifying an antibody in the set of antibodies for subsequent production includes identifying an antibody that is thermostable and manufacturable.

In some embodiments, identifying a subset of the antibodies involves determining whether the thermostability indications satisfy one or more criteria. For example, antibodies whose thermostability indications comprise a temperature that exceeds a particular threshold may be identified. As another example, antibodies whose thermostability indications include a particular range of temperatures may be included. When the thermostability indication determined for a particular antibody satisfies one or more criteria, that antibody may be included in the subset of antibodies for subsequent production. If the thermostability indication does not satisfy the one or more criteria, the antibody may be excluded from the subset of antibodies for subsequent production.

As described above, in some embodiments, act 504 may be performed using a computing device (e.g., computing device(s) 120 shown in FIG. 1B). Additionally, or alternatively, act 504 is performed by a user. For example, a user may manually select antibodies for subsequent production based on the thermostability indications output by the trained machine learning model.

In some embodiments, the identified subset includes none, some, or all of the antibodies included in the original set of antibodies. For example, the identified subset may include 0%, less than 10%, less than 25%, less than 50%, less than 75%, less than 90%, or all of the antibodies included in the original set of antibodies.

Process 500 then proceeds to act 506, where at least some of the antibodies included in the identified subset of antibodies are produced. In some embodiments, the antibodies are produced using techniques known in the art.

In some embodiments, producing at least one of the antibodies in the subset of antibodies includes producing one, some, or all of the antibodies in the subset. For example, in some embodiments, at least 10%, at least 25%, at least 50%, at least 75%, at least 90%, or all of the antibodies included in the identified subset are produced at act 506.

In some embodiments, implementing process 500 may include additional or alternative steps that are not shown in FIG. 5A. In some embodiments, process 500 may include only a subset of the acts included in the example flowchart (e.g., act 502 only, acts 502 and 504 only).

FIG. 5B is a flowchart of an illustrative process 550 for determining thermostability for a first antibody, in accordance with some embodiments of the technology described herein. In some embodiments, act 502 of process 500 may implemented using process 550. Process 550 may be performed by any suitable computing device(s) (e.g., computing device(s) 120 shown in FIG. 1B).

Process 550 begins at act 552, where information indicative of a 3D structure of the first antibody is obtained. In some embodiments, this information was previously-obtained for the first antibody. Thus, in some embodiments, obtaining the information indicative of the 3D structure of the first antibody may include accessing the information (e.g., from a memory, over a network, via a file being provided via an appropriate interface, etc.).

In other embodiments, obtaining the information indicative of the 3D structure of the first antibody comprises generating this information. Accordingly, in some embodiments, obtaining the information indicative of the 3D structure of the first antibody includes generating that information by processing the residue sequence of the first antibody using protein structure prediction software. The protein structure prediction software may be configured to output the information indicative of the 3D structure of the first antibody. Any suitable protein structure prediction software may be used. Examples of protein structure prediction software are described herein including at least with respect to act 252 of FIG. 2B.

Process 550 then proceeds to act 554, where interaction energy metrics for each of a plurality of pairs of amino acid residues of the first antibody are obtained. In some embodiments, obtaining the interaction energy metrics includes generating the energy metrics by processing the information indicative of the 3D structure of the first antibody using molecular modeling software. The molecular modeling software may be configured to output the interaction energy metrics for the first antibody. Any molecular modeling software capable of estimating residue interaction energy metrics may be used. Examples of molecular modeling software are described herein including at least with respect to act 254 of FIG. 2B.

In some embodiments, interaction energy metrics are obtained for some or all of the pairs of residues of the residue sequence of the first antibody. For example, interaction energy metrics may be obtained for at least 75%, at least 80%, at least 85%, at least 90%, at least 95%, at least 98%, at least 99%, or all of the pairs of residues of the amino acid sequence of the first antibody.

Any of numerous types of interaction energy metric(s) may be obtained for a pair of residues. Examples of interaction energy metrics are described herein including at least with respect to act 254 of FIG. 2B.

Process 550 then proceeds to act 556, where a first set of features is generated for the first antibody so that the first set of features may be provided as input to the trained machine learning model at act 558 to obtain a corresponding output indicative of a first thermostability for the first antibody. In some embodiments, generating the first set of features involves including, in the first set of features, at least some of the data obtained at act 552 and/or act 554 of process 550.

For example, at act 556a, interaction energy metrics are included in the first set of features. In some embodiments, the interaction energy metrics include some or all of the interaction energy metrics obtained at act 554, examples of which are provided herein.

In some embodiments, including the interaction energy metrics in the first set of features at act 556a includes generating one or more matrices of interaction energy metrics. For example, for each particular energy metric, a respective 2D matrix may be generated that has, in its entry in the ith row and jth column, the value of that particular metric for the ith and jth residues. When the interaction energy metrics include multiple metrics, a 2D matrix may be generated for each of them and therefore included as part of the first set of features being generated at act 556a of process 550. The 2D matrices may be therefore provided as inputs (e.g., via different channels as shown in FIG. 8) to the trained machine learning model. Alternatively, a 3D matrix may be generated instead of multiple 2D matrices, as aspects of the technology described herein are not limited in this respect.

Additionally, or alternatively, at act 556b, an encoded first residue sequence is included in the first set of features. In some embodiments, encoding the first residue sequence to obtain the encoded first residue sequence may be performed using any suitable encoding technique, such as one-hot encoding. In some embodiments, the first residue sequence is encoded to form a particular input dimension. For example, the first residue sequence may be encoded to form an input dimension of (V_{H}+V_{L}+3)×21, where V_{H} and V_{L} correspond to the heavy and light chain sequences of the antibody residue sequence, respectively.

Though not shown in FIG. 5B, it should be appreciated that one or more additional or alternative features may be included in the first set of features, as aspects of the technology described herein are not limited in this respect.

Next, process 550 proceeds to act 558, where the first set of features is provided as input to the trained machine learning model to obtain an output indicative of the thermostability of the first antibody.

In some embodiments, the machine learning model is of any suitable type. For example, the machine learning model may be a neural network, such as a Convolutional Neural Network (CNN). The CNN may have one or more two-dimensional convolutional layer, one or more three-dimensional convolutional layer, and/or a fully connected layer.

As one specific example, the CNN may have the architecture shown in the FIG. 8, which includes one or more max pooling layers (Adaptive MaxPool 1D and Adaptive Max Pool2D layers in FIG. 8), one or more 2-dimensional convolution layers, one or more non-linearity layers (e.g., a rectified linear unit layer “ReLu” in FIG. 8), one or more batch normalization layers, and a dense layer. The architecture shown in FIG. 8 shows that both energy metrics and one-hot encoded sequences are provided as input. However, as described herein, energy metrics only or one-hot encoded sequences only may be provided as input, in some embodiments. Thus, in some embodiments, either the top branch or the lower branch or both branches of the architecture shown in FIG. 8 may be used.

As another example, the machine learning model may be a pre-trained language model (used either to perform zero-shot predictions or tuned by adding a supervised head using transfer learning with a small amount of training data), such as a bidirectional encoder representations from transformers (BERT) model (e.g., an ESM-1b language model or the ESM-1v language model) or a UniRep language model, which are described herein. Such models may be used when the input only includes sequence features and does not include energy features (e.g., as shown in FIG. 14, for example).

In some embodiments, the machine learning model includes multiple machine learning models (e.g., an ensemble of machine learning models). For example, the machine learning model may include an ensemble of neural networks. In some embodiments, implementing an ensemble of machine learning models includes training each of multiple (e.g., two or more, three or more, etc.) machine learning models on different training datasets, predicting thermostability using each of the trained machine learning models, and averaging (e.g., weighted averaging of) their predictions. Outputs of multiple machine learning models may be combined in any other way, as aspects of the technology described herein are not limited in this respect. The ensemble of machine learning models may be obtained, in part, by using any suitable bagging or boosting technique.

In some embodiments, the machine learning model is trained to process the first set of features to obtain the probability that the thermostability of the first antibody belongs to each of multiple classes of thermostabilities. For example, this may include determining the probability that the first antibody is thermostable in each of a plurality of temperature ranges. As a nonlimiting example, classes of thermostabilities may include the following four classes: under-50° C., 50° C.-60° C., 60° C.-70° C., and 70° C.-up.

In some embodiments, based on the determined probabilities, the machine learning model is configured to predict a class of the multiple classes associated with the highest probability. For example, this may include identifying a temperature range of multiple temperature ranges associated with the highest probability. The identified temperature range includes one or more temperatures at which the first antibody is thermostable.

Additionally, or alternatively, based on the determined probabilities, the machine learning model may be configured to determine a temperature at which the first antibody is thermostable. For example, the temperature may be the TS50 temperature. As another example, the temperature may correspond to the thermal melting temperature (Tm.) In some embodiments, determining the temperature at which the first antibody is thermostable includes determining, as the temperature, a weighted linear combination of the temperature range means weighted by the probabilities determined using the machine learning model.

At act 560, process 550 includes determining whether there is another antibody in the set of antibodies for which thermostability can be determined. When it is determined, at act 560, that there is another antibody for which thermostability is to be determined, acts 552-558 are repeated for the other antibody. For example, for a second antibody, this would include determining a second set of features and providing the second set of features as input to the trained machine learning model to determine a thermostability indication for the second antibody.

### EXAMPLES

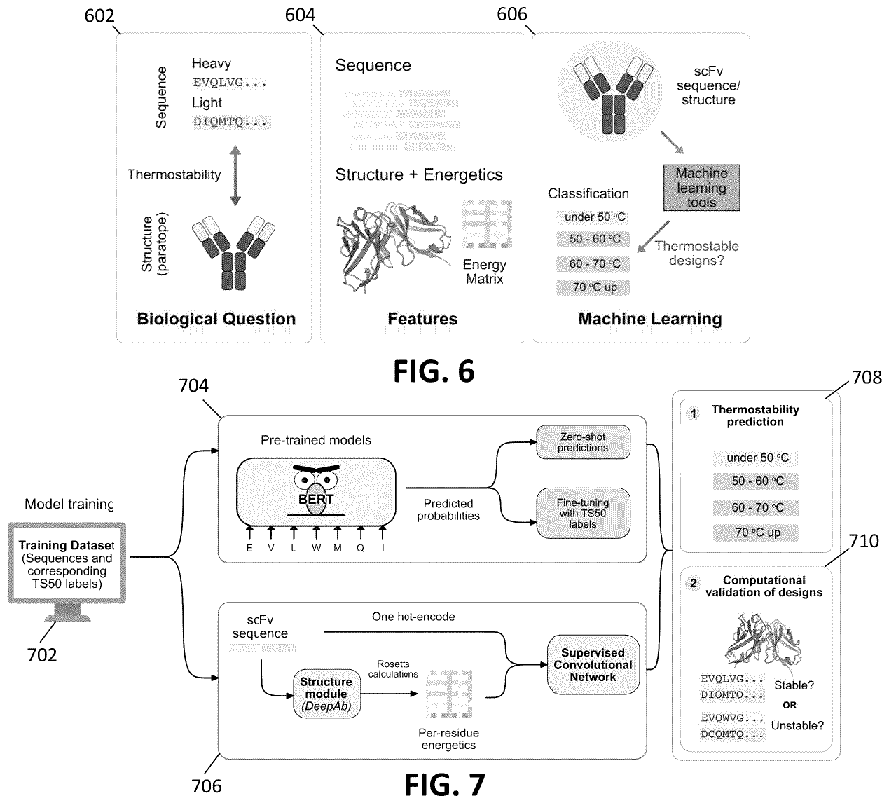

Machine learning techniques were developed for predicting thermostability of scFvs. In some embodiments, the developed machine learning techniques utilize different features, generated for the scFv, to predict its thermostability. FIG. 6 is a diagram of an example of an scFv 602, a set of features 604 that may be generated for the scFv 602, and a machine learning model 606 trained to predict thermostability for the scFv 602.

FIG. 7 is a diagram of an example technique for training and using various machine learning models to predict thermostability of scFvs on datasets (e.g., labelled TS50 dataset) 702. Branch 704 shows transfer learning with an unsupervised network, such as a pre-trained language model (PTLM), that can be used to predict thermostability with zero-shot and fine-tuned predictions. Branch 706 shows a supervised model, such as a neural network architecture (e.g., a supervised convolutional neural network (CNN)), that is trained to predict thermostability of an scFv using features derived from the scFv. Both types of models may be employed to predict thermostability 708, to computationally validate experimental designs 710, or for any other suitable purpose, as aspects of the technology described herein are not limited in this respect.

A. Supervised Neural Network

FIG. 8 is a diagram showing an example architecture of a supervised convolutional neural network (CNN) trained to predict thermostability of an scFv using sequence and/or energy features generated for the scFv. The parameters of the example model were estimated with ADAM optimizer with categorical cross entropy (CCE) loss and a learning rate of 10⁻³. The model was trained using the datasets described here including at least in the section “Datasets.”

As shown in FIG. 8, the input scFv sequences 802 are processed using protein structure prediction software 804 (e.g., DeepAb, AlphaFold, etc.) to generate information indicative of the 3D structure of the scFv. The structure information is used to evaluate thermodynamic features 806 (total energy split into one-body i-I, and two-body, i-j, residue energies) for each scFv, using Rosetta ref2015 energy function. The contributions of the ith residue with every jth residue (where j∈N such that N=total number of residues) may be tabulated and classified in an i-j matrix that constituted the energy features.

Additionally, or alternatively, as shown in FIG. 8, the input scFv sequences 802 are encoded (e.g., one-hot encoded) to obtain sequence features 810.

The energy features 806 and sequence features 810 are converted to a fixed length embedding of size L×L and L, respectively, where L represents the maximum sequence length in the dataset, such that V_{H} and V_{L} are the maximum lengths of the heavy and light chains, respectively. Input scFv sequences 802 less than L are padded with zeros.

The energy features 806 and sequence features 810 are provided as input to two parallel branches of the model. In particular, the energy features 806 are provided as input to a 2D convolutional layer 808 and the sequence features 810 are provided as input to a 1D convolutional layer 812. For example, the sequence and energy features may pass through respective convolutional layers with Batch Normalization and ReLU activation.

As shown in FIG. 8, the sequence input is transformed and concatenated with the energy input. The concatenated matrix is then passed through another 2D convolutional layer 814, flattened, and supplied to a dense layer 816 to output logits for each class. The class probabilities 818 may be obtained by performing a normalized exponential function over the logits.

In some embodiments, the class probabilities 818 represent the probability that thermostability (e.g., the TS50 measurement or Tₘ measurement) of the scFv corresponds to a particular temperature range.

To estimate the predicted thermostability (e.g., TS50 value or Tm value) value, the probabilities are weighed with the mean thermostability (e.g., mean TS50 or mean Tm) value in each class.

Thermo_{predicted}(x)=Σᵢ₌₁ⁿ⁼⁴p(x).Thermo_{bin(i)} (Equation 1)

Additionally, or alternatively, the techniques may include classifying the scFv into the class corresponding to the highest probability output by the machine learning model. The identified class may correspond to a range of temperatures including at least one temperature at which the scFv is thermostable.

In some embodiments, the architecture shown in FIG. 8 may be used to predict thermostability using only one of the energy features 806 and sequence features 810 as input. In this case, the architecture of the model may not be altered. Rather, a tensor of zeros may be provided as input in place of one of the features. For example, to use only the energy features 806 to predict thermostability, a tensor of zeros may be passed through the top branch of the architecture shown in FIG. 8, as opposed to the sequence features 810. Similarly, to use only the sequence features 810 to predict thermostability, a tensor of zeros may be passed through the bottom branch of the architecture shown in FIG. 8, as opposed to the energy features 806.

Experiments were performed to evaluate the performance of various machine learning models (e.g., having the architecture shown in FIG. 8) in predicting thermostability on the datasets described herein, including with respect to the section “Datasets”. The “energetics-only” model was configured to predict thermostability using only energy features (e.g., energy features 806) as input. The “sequence-only” model was configured to predict thermostability using only sequence features (e.g., sequence features 810) as input. The “energetics +sequences” model was configured to predict thermostability using both sequence and energy features (e.g., energy features 806 and sequence features 810).

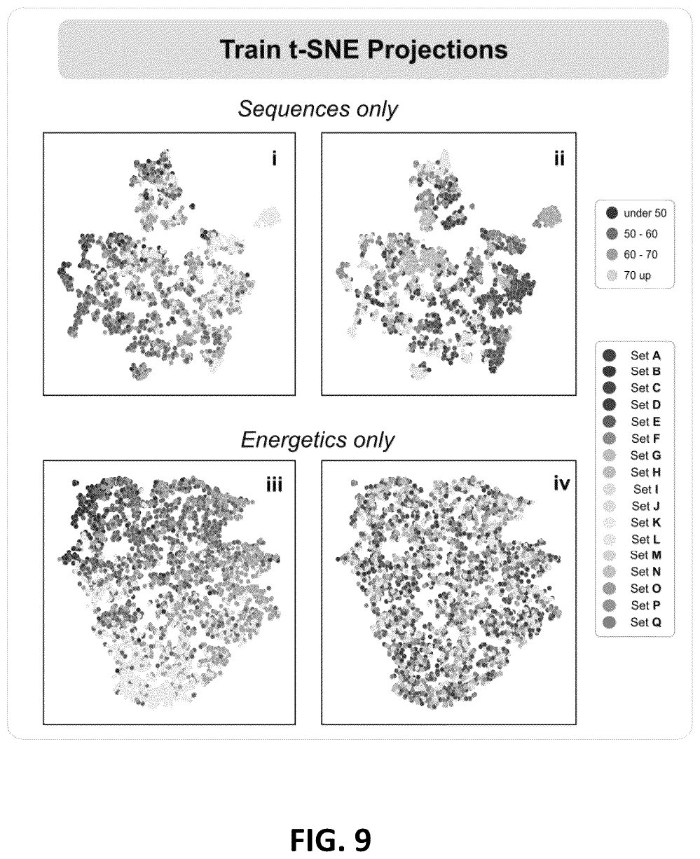

The outputs of the models were used to evaluate whether the experimental sets from which the scFvs were derived had an impact over prediction accuracy. By projecting the embeddings from the dense layer for each sequence into two dimensions via t-distributed stochastic neighbor embedding (t-SNE), the representation learned by the sequence-only model and the energetics-only model can be analyzed. FIG. 9 shows t-SNE generated for each model. The sequence-only model embeddings were clustered by their experimental set, as evident by the aggregation of shaded points FIG. 9. The energetics-only model embeddings were independent of any clustering based on the experimental set as demonstrated by the noisy embedding for energetics. Thus, in spite of a sequentially diverse dataset, fine-tuned and supervised models trained only on sequence-features are able to infer the underlying experimental origin of the sequences and skew thermostability predictions, making them less generalizable towards newer, blind datasets. By contrast, the energetics-only model is more generalizable towards newer, blind datasets.

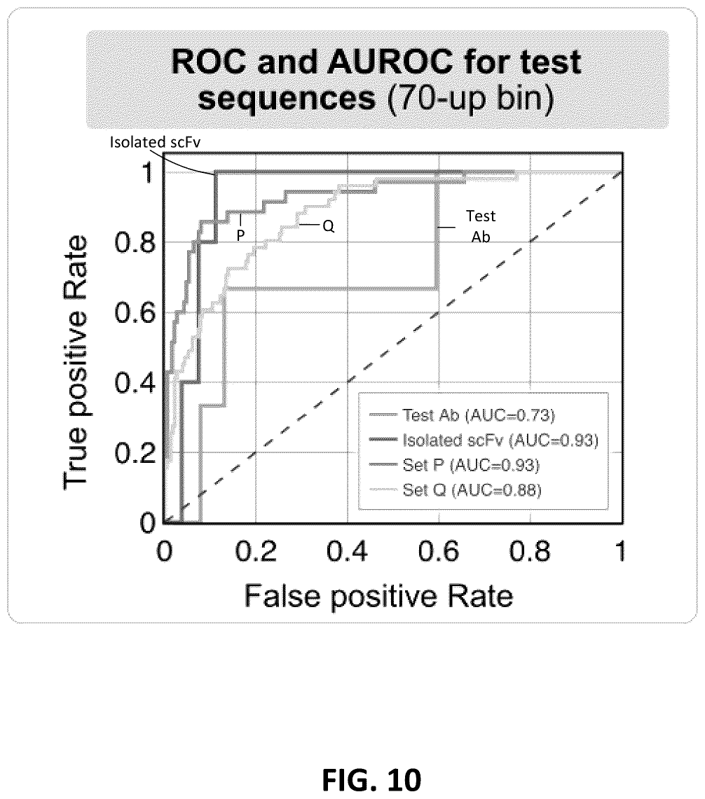

The performance of the energetics-only model was assessed by constructing a receiver-operating-characteristic (ROC) curve derived from the prediction of the 70° C.-up class. FIG. 10 shows the constructed ROC curve. The ROC was evaluated for four test datasets: two held-out (Set P and Set Q) and two blind datasets representing a test antibody (Test Ab) and an isolated scFv (Isolated scFv). The area under ROC is over 0.7, denoting a high classification accuracy.

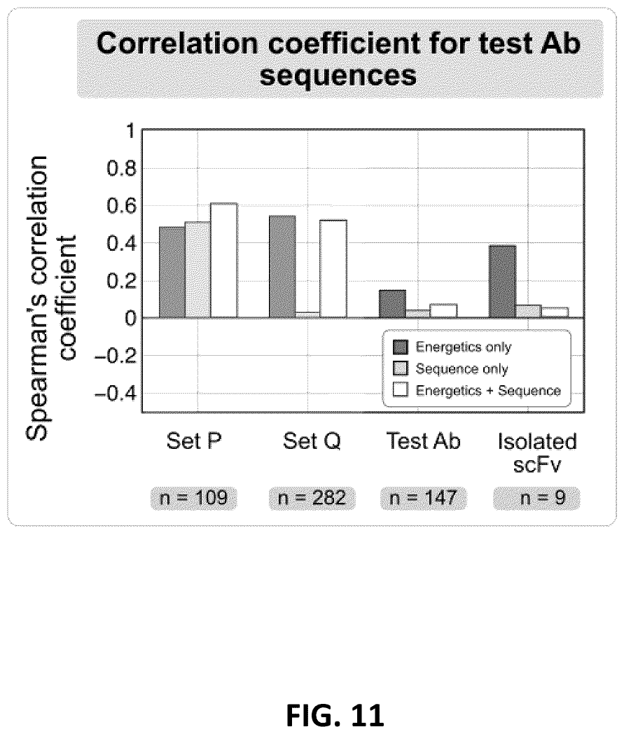

FIG. 11 shows a graph comparing performance of the various machine learning models for predicting thermostability of scFvs. In particular, FIG. 11 shows the correlation coefficient for all four test datasets, with the energetic-only, sequence-only and energetics+sequences models, respectively. On held-out datasets, the coefficients are over 0.5 for energetics-only model with energetics+sequences model showing an equally improved performance. But on blind datasets, the performance drops for energetics+sequences and sequences-only (coefficients under 0.1). The energetics-only model still shows relatively high correlation for the blind datasets (0.2 and 0.4 respectively).

As a control, weights were randomly initialized in the S-CNN for the classification task. The results showed that it is unable to distinguish sequences based on thermostability. Further, on the test sets, weighted random predictions were performed i.e., predicted the class label with a weighted random choice, with sample size in each class as the weights. In both these tests, the energetics-only S-CNN was able to decipher some relationship between the energetics of the scFv and the thermostability. The randomly initialized models could not demonstrate any discernible relationship demonstrating the significance of learned representations from supervised data.

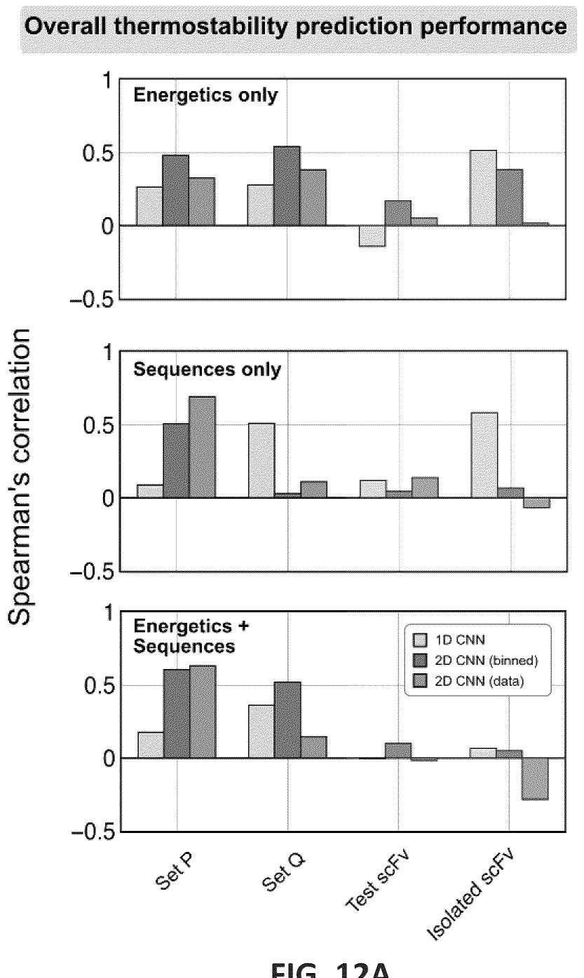

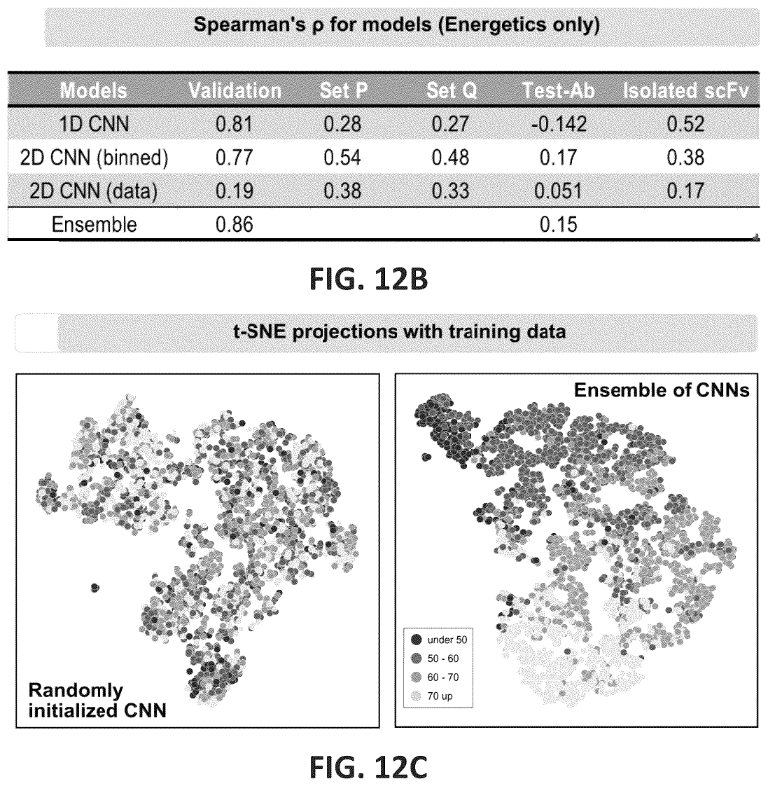

As described herein, FIG. 8 shows one example supervised CNN architecture that may be used to predict thermostability on sequence and/or energy features. In the embodiment shown in FIG. 8, the 2D-CNN classification model is used for the energy features. However, there may be one or more alternative ways to feed the energy features, such as, for example, a 1D flattened input or a 2D input with absolute residue-wise energy values. These architectures were tested, and their performance compared, to inform architecture-selection. The results are shown in FIG. 12A. For the energetics-only case, the 2D-CNN with classified inputs showed improved performance compared to the other two architectures.

Additionally, or alternatively, to reduce variance of model performance, the performance of multiple models may be ensembled by averaging their predictions and generating an ensemble of CNNs. Three supervised CNNs, having the same architecture, were trained on different dataset splits. The experimental sets were shuffled to obtain three different pairs of training and held-out data. FIGS. 12B and 12C demonstrate improved performance using the ensemble of CNNs to predict thermostability, as compared to the performance to the non-ensembled CNNs.

In order to test a control, a randomly initialized CNN was employed and compared the embeddings generated with this randomized model with the ensemble of CNNs. As shown, the model developed by the inventors can better separate the sequences based on their thermostability characteristics, even with limited sequence size and training diversity.

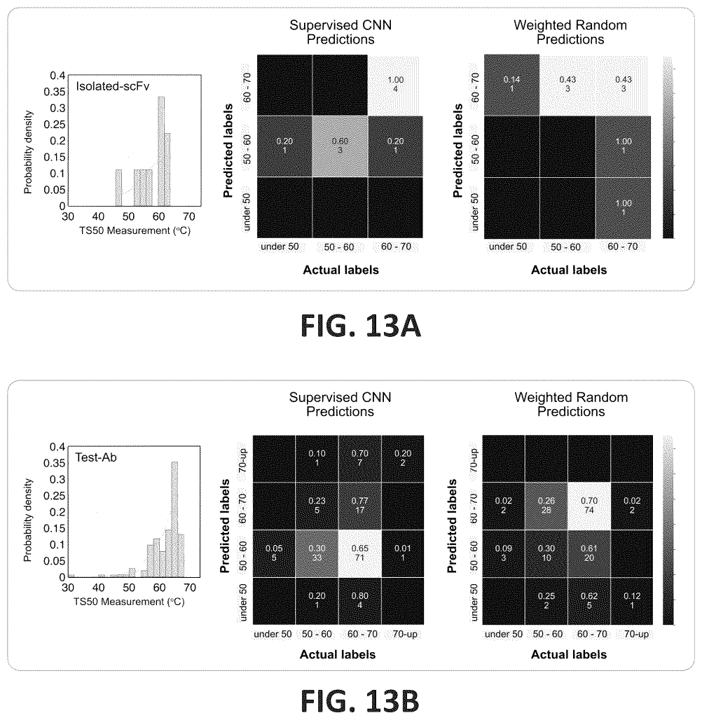

The performance of the ensemble of CNNs was also tested on the blind datasets i.e., the test scFv and the isolated scFv sequence with respect to a weighted random prediction model. For this case, the randomization was biased with the weights of the sample size of each class as observed from the training dataset. This sample size was then used as a probability with the random number generator to predict the classes of the sequences. While the data points are limited, non-uniform and heavily skewed, FIGS. 13A-13B show that the thermostability predictions obtained using the machine learning techniques developed by the inventors can be used to observe trends in thermostability of scFvs, as opposed to the weighted random predictions. In particular, FIGS. 13A-13B show confusion matrices highlighted with the probabilities of the prediction in each class. The predictions in the top-most class are skewed more towards the higher temperature regions in the machine learning predictions as opposed to the weighted random predictions. This implies that, while predicting blind sequences, if the top-most class i.e., 70-up class, is considered, then there is a higher probability of actually selecting sequences which are thermostable i.e., lie in the top 2 classes, 60-70 or 70-up. This is important as it can help remove redundant, potentially less thermostable sequences and with well-curated training sets, simple supervised networks could be useful for making robust design estimations.

B. Pre-Trained Language Model

Pre-trained language models (PTLMs) were evaluated to assess their ability to predict thermostability of scFv sequences. FIG. 14 is a diagram depicting an example technique 1400 for predicting thermostability of an scFv using a PTLM, in accordance with some embodiments of the technology described herein. The pre-trained language models 1402 were evaluated to assess their ability to predict thermostability using zero-shot predictions 1404 and fine-tuned predictions 1406.

Three pre-trained language models were evaluated. The first model, UniRep, is an mLSTM with 1900 hidden units pretrained on the Pfam database. Multiple sequence alignments (MSAs) were collected for each sequence in the TS50 set, as proposed in the “evotuning” approach described by A Bateman et al., “The pfam protein families database.” Nucleic acids research (2004), which is incorporated by reference herein in its entirety. The sequences were combined into a single dataset, and the model was pretrained on this evolutionarily related set of sequences using the implementation described by Ma, Eric J., and Arkadij Kummer. “Reimplementing UniRep in JAX.” bioRxiv (2020), which is incorporated by reference herein in its entirety.

Additionally, both the ESM-1b and ESM-1v transformer models were considered. Both are 33-layer, 650 M parameter transformer models, pretrained with masked language modeling on the Uniref database. ESM-1b is trained on a 50% sequence identity filtered dataset (Unired50), while ESM-1v is trained on a 90% sequence identity filtered dataset (Unifer90). The ESM-1b transformer model is described by A Rives, et al., “Biological structure and function emerge from scaling unsupervised learning to 250 million protein sequences.” Proc. Natl. Acad. Sci. (2021), which is incorporated by reference herein in its entirety. The ESM-1v transformer model is described by J Meier, et al., “Language models enable zero-shot prediction of the effects of mutations on protein function.” Adv. Neural information processing systems (2021), which is incorporated by reference herein in its entirety.

i. Zero-Shot Evaluation

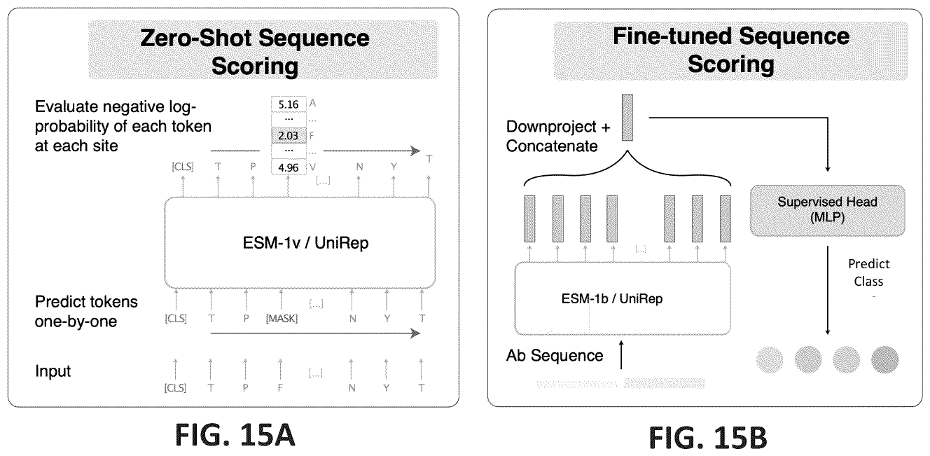

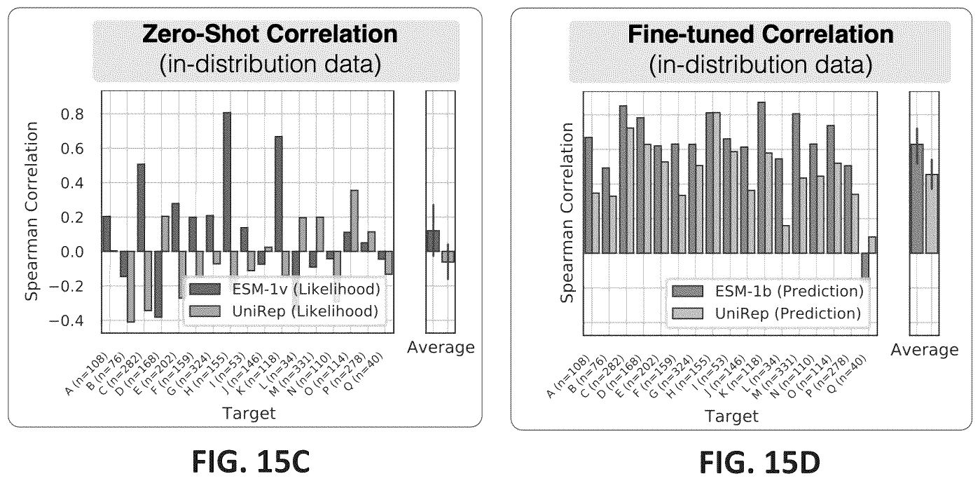

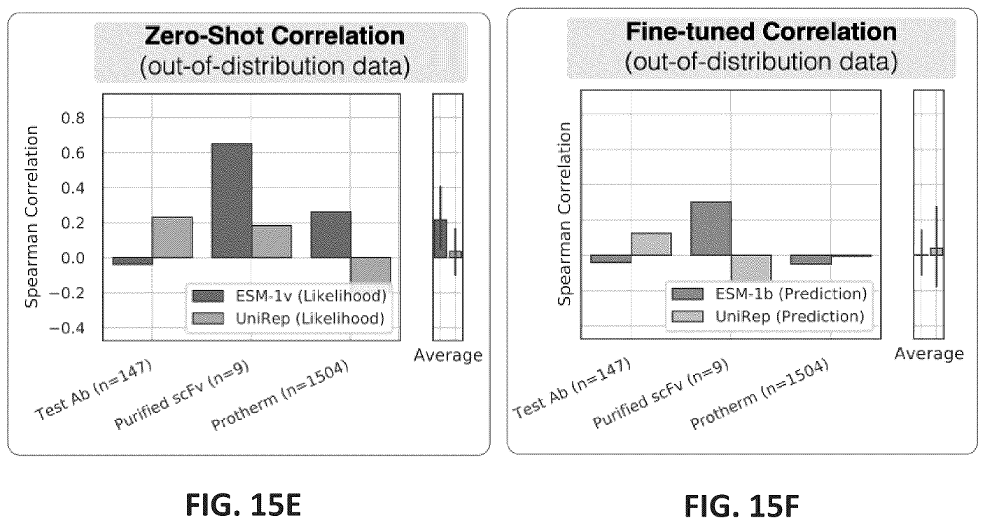

FIG. 15A is a diagram of an example pre-trained language model configured to make zero-shot thermostability predictions using sequence data, in accordance with some embodiments of the technology described herein.

One approach to predicting thermostability with pretrained language models is to directly use model likelihood or pseudolikelihood. Sequences which are more likely under a model are predicted to be more thermostable. UniRep models the probability of each residue of a residue sequence given all preceding residues. As a result, the likelihood of a sequence can be efficiently evaluated as:

[symbol]

_{UniRep}(x)=Πᵢ₌₁ⁿp(xᵢ|xⱼ∀j<i) (Equation 2)

ESM-1v models the probability of masked residues given unmasked residues. The pseudo-likelihood of a sequence can be obtained as:

pseudo [symbol] _{ESM-1v}(x)=Πᵢ₌₁ⁿp(xᵢ|xⱼ∀j≠i) (Equation 3)

In practice, the log-likelihood and pseudo-log-likelihood are evaluated for numerical stability. Additionally, ESM-1v comes as an ensemble of five models trained with different random seeds. The predictions from all five models are averaged to obtain the final pseudo-log-likelihood.

ii. Finetuned Evaluation

FIG. 15B is a diagram of an example fine-tuned pre-trained language model configured to make thermostability predictions using sequence data, in accordance with some embodiments of the technology described herein.

For the UniRep model the final hidden state is taken along with the average of previous hidden states as a fixed-length vector representation of 3900 hidden units. For the ESM-1b model, each per-residue representation is down projected to 4 dimensions, followed by a concatenation. This results in a fixed length embedding of size 4L where L is the maximum sequence length in the TS50 dataset. If a sequence has length less than L, it is padded with zeros.

These embeddings are passed through a linear layer with a hidden dimension of 512, followed by tan h activation, then to a final layer to predict class logits. Parameters of the UniRep and ESM-1b models are frozen during training. Parameters of the head model (including the initial down projection for ESM-1b) are trained with the Adam optimizer and a learning rate of 10⁻³.

For TS50 data, models were trained on all but one target and evaluations are made on the held-out target. For non-TS50 data, an ensemble of TS50 models (one for each holdout target) is used to make predictions.

iii. Results

FIGS. 15C, 15D, 15E, and 15F are graphs showing that fine-tuned predictions achieve improved correlation with thermostability as compared to zero-shot predictions, in accordance with some embodiments described herein.

In particular, FIG. 15C and FIG. 15E show that zero-shot predictions do not generally correlate well with thermostability, either on the TS50 datasets described in the section “Datasets” or on blind test sets. By contrast, FIG. 15D shows that the fine-tuned predictions from both ESM-1b and UniRep achieved moderate to high average Spearman correlation on held-out targets when trained on TS50 datasets (0.63 and 0.45). However, as shown in FIG. 15E, these predictions did not generalize well to blind test sets. This suggests there is some underlying structure in the sequences in the TS50 dataset that the model can exploit to make predictions, but which did not generalize to the new datasets.

C. Comparing Performance of Machine Learning Models

Experiments were performed to evaluate the ability of the supervised models and the pre-trained learning models, described herein, in discriminating between thermostable and thermally degenerate mutations.

Thermal aggregation experiments on point mutations for an anti-VEGF antibody (PDB ID: 2FJG/2FJF) are detailed in studies by P Koenig et al. “Mutational landscape of antibody variable domains reveal a switch modulating the interdomain confirmational dynamics and antigen binding,” Proc. Natl. Acad. Sci. United States Am. (2017), and S Warszawski et al., “Optimizing antibody affinity and stability by the automated design of the variable light-heavy chain interfaces,” PLoS Comput. Biol. (2019), each of which is incorporated by reference herein in its entirety. In both studies, a deep mutational scanning (DMS) experiment was performed for the antibody and point mutations that improved binding enrichment over wildtype were analyzed for their fragment antigen-binding (Fab) melting temperature (Tₘ). These point mutants (20 mutations compiled from both the studies) serve as a test case to evaluate whether the networks trained on TS50 temperature measurements could obtain insights about related temperature-dependent attributes such as thermal aggregation; and whether they distinguish the thermally-enhancing and thermally-hampering mutations.

To determine whether the predictive models described herein have potential in protein design, a computational DMS was created on the anti-VEGF antibody (PDB ID: 2FJG). Each residue position in the sequence was mutated to 19 other amino acids to obtain mutant sequences. Each sequence was one-hot encoded to obtain the sequence data, and the energetics dataset was generated (e.g., according to the techniques described herein including at least with respect to FIG. 2B). This sequence and energy input was fed to the models and the point mutants classified in the 70-up temperature class by the machine learning predictions were cross-verified with the experimental results.

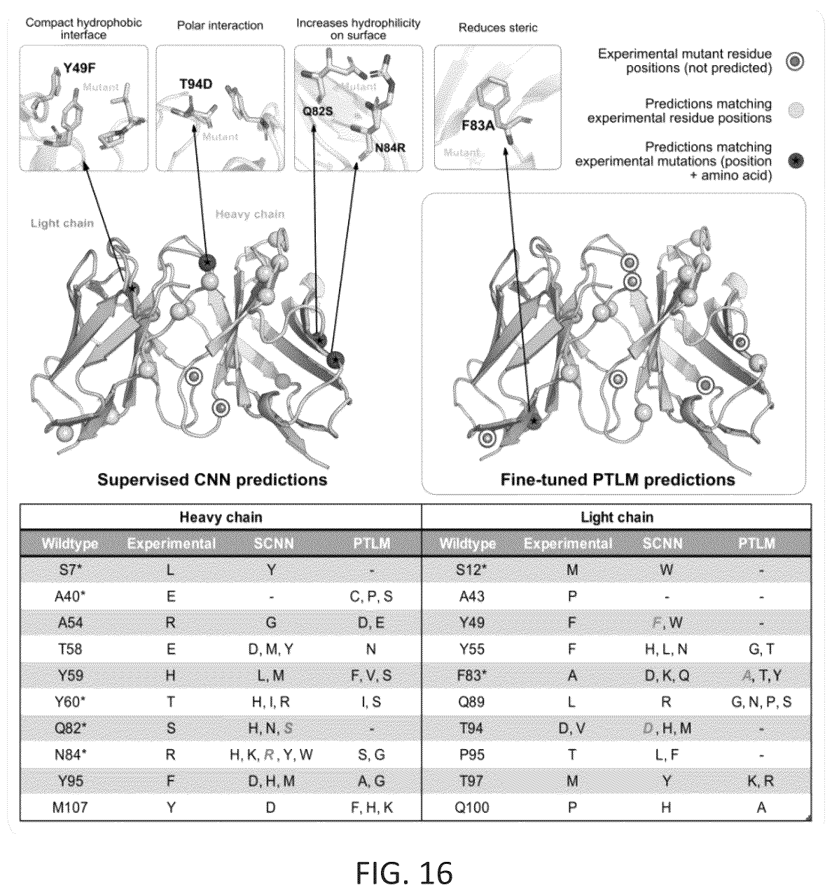

FIG. 16 is a diagram showing that the thermostability predictions determined using the machine learning techniques, in accordance with some embodiments of the technology described herein, agree with the experimentally-determined thermostabilities. The spheres, shown in FIG. 16, indicate the experimentally validated mutants that improved thermostability (e.g., Tₘ) of the scFv. The starred spheres indicate that the machine learning techniques accurately predicted the mutation and residue position that result in the most thermostable scFvs. The light grey spheres indicate that the machine learning techniques accurately predicted the residue position, but not the mutation, which results in the most thermostable scFvs. The ringed spheres indicate mutations which were not observed using the machine learning techniques.

As shown, the CNNs were able to identify five out of the 20 mutations correctly. Further, for 18 out of 20 mutations, the CNNs could identify the residue position correctly, albeit predicting different amino-acid mutations as most thermostable. Out of 4,540-point mutations analyzed (Nᵣₑₛ=227 residues, 20 amino acids per residue), experimental data was available for only 20 point mutations. Since only 0.44% of the total possible mutations in the anti-VEGF antibody were assessed for melting temperatures experimentally, the validation dataset for thermostability is scarce. Further, in spite of being temperature-specific attributes, TS50 and Tₘ are different experimental measurements and do not correlate exactly. It is, therefore, remarkable that the CNNs could predict the thermostable residue positions in 90% of the cases, with 25% successful predictions (correct residue positions as well as amino-acid residues). By extrapolating the networks trained on TS50 measurements over alternative thermal aggregation experiments (Tₘ in this case), it is demonstrated that intrinsic thermal attributes could be captured by such models.

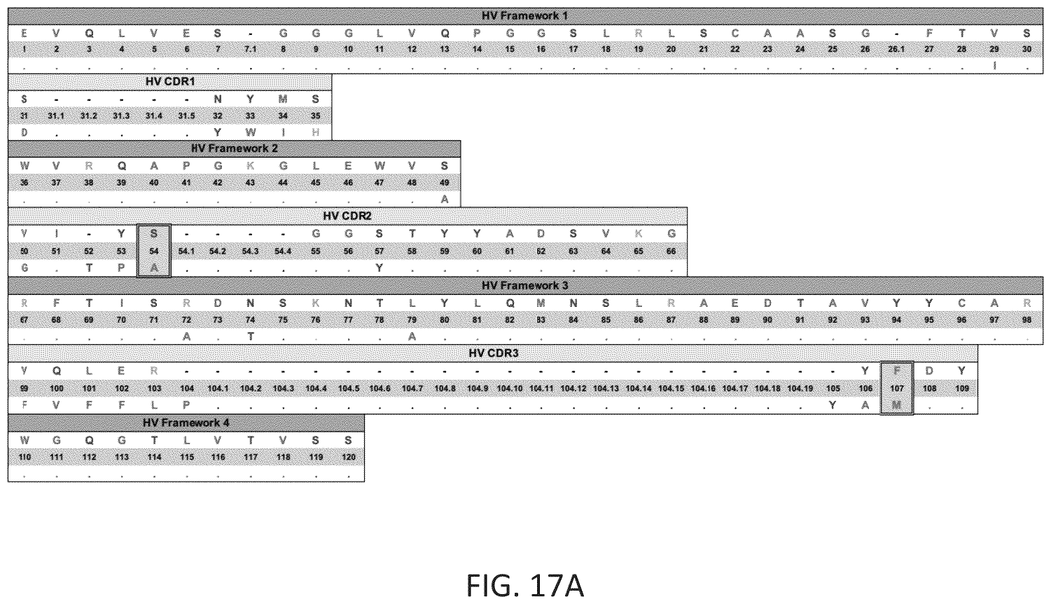

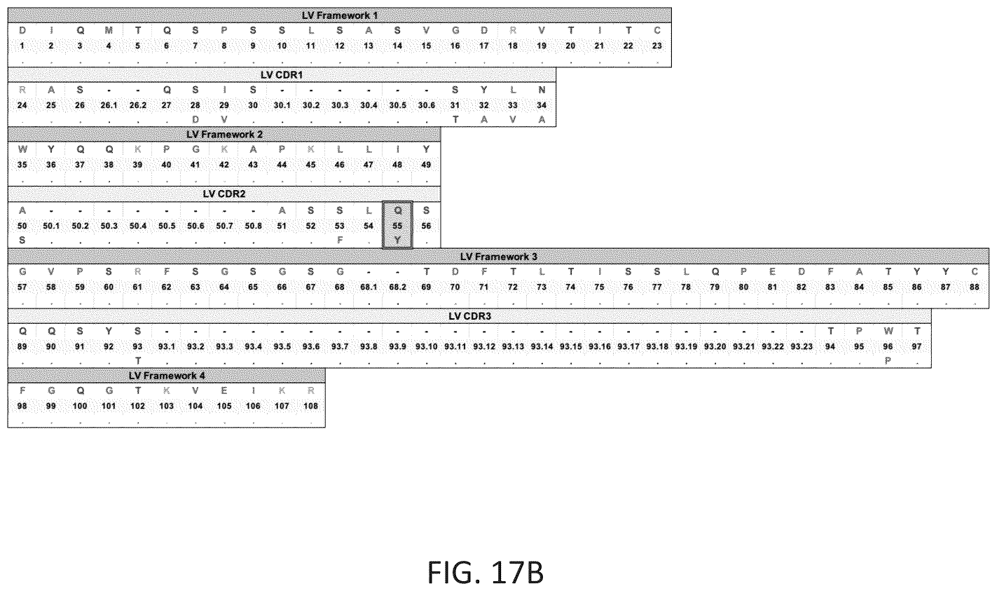

Moreover, on comparing the residue positions violating the germline consensus sequence for the anti-VEGF Ab, as shown in FIGS. 17A-17B, different amino acid mutations were observed, highlighting the ability of the machine learning techniques described herein to provide mutations orthogonal to traditional germ lining approaches. With a more diverse and larger training dataset, it would be possible to develop a more robust model. The results suggest that these networks could serve as a useful tool for screening or filtering antibody sequences for temperature-specific antibody design pipelines.

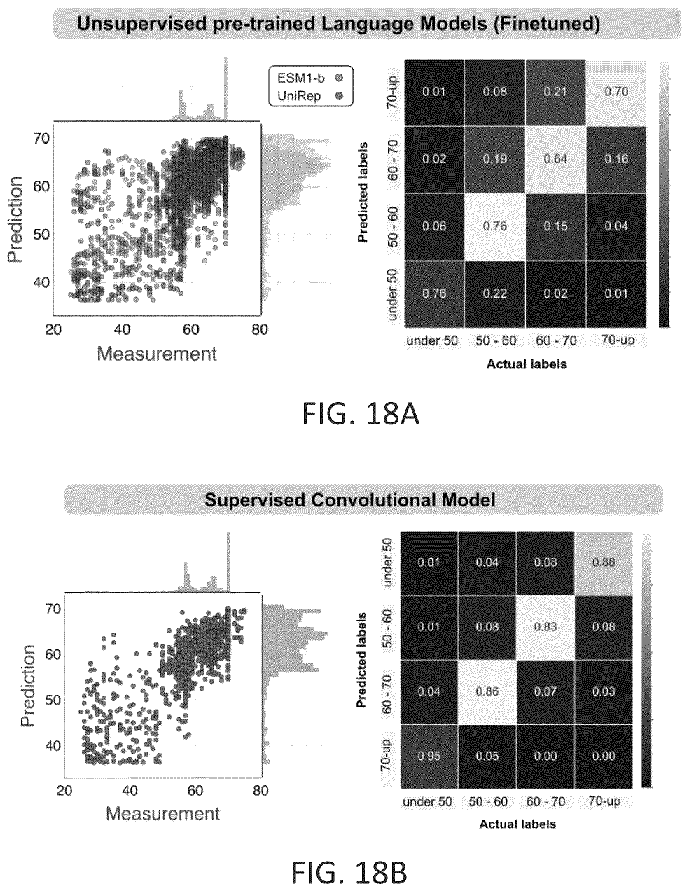

FIGS. 18A-18B are graphs showing that thermostability predictions output by a supervised convolutional neural network achieve improved correlation with thermostability as compared to thermostability predictions by an unsupervised pre-trained language model, in accordance with some embodiments of the technology described herein.

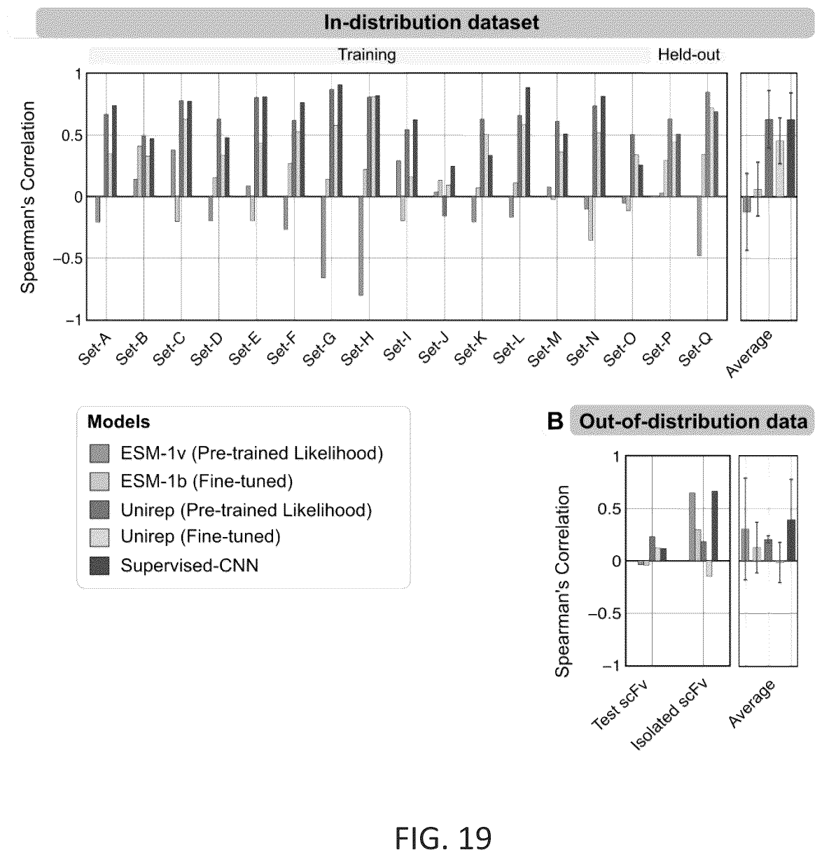

FIG. 19 shows graphs comparing performance of the various machine learning models described herein for predicting thermostability of scFvs, in accordance with some embodiments of the technology described herein. As shown, training and prediction using the supervised-CNN model is generally more accurate than training and predication using the pre-trained language models.

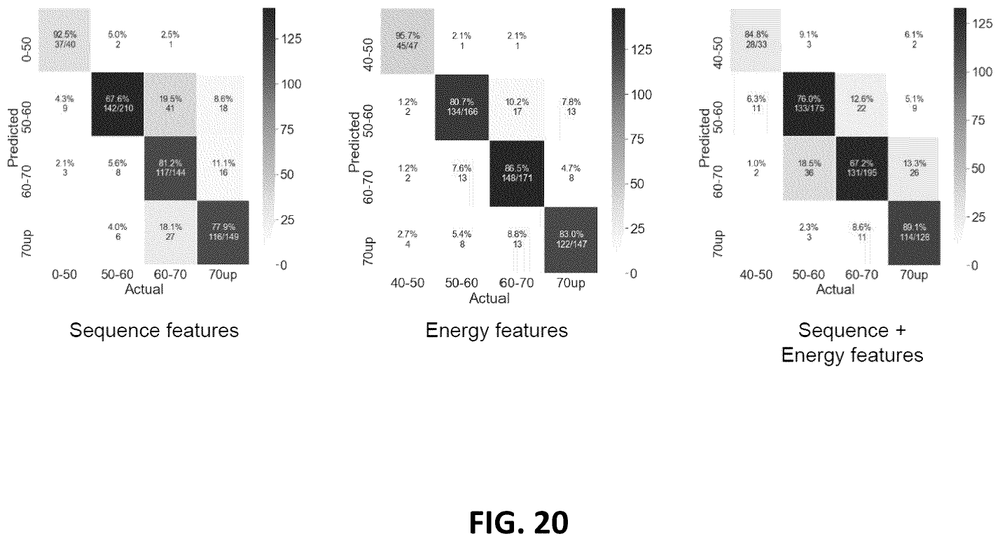

FIG. 20 includes graphs showing that training and prediction based on residue-pair interaction energy metrics is more accurate than training and prediction based on encoded sequences and training and prediction based on both encoded sequences and residue-pair interaction energy metrics, in accordance with some embodiments of the technology described herein.

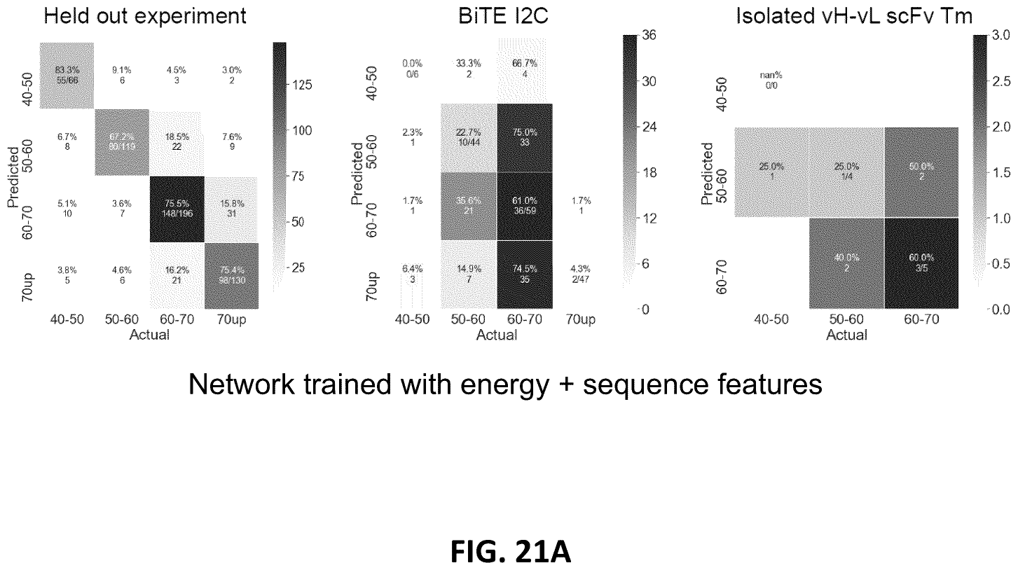

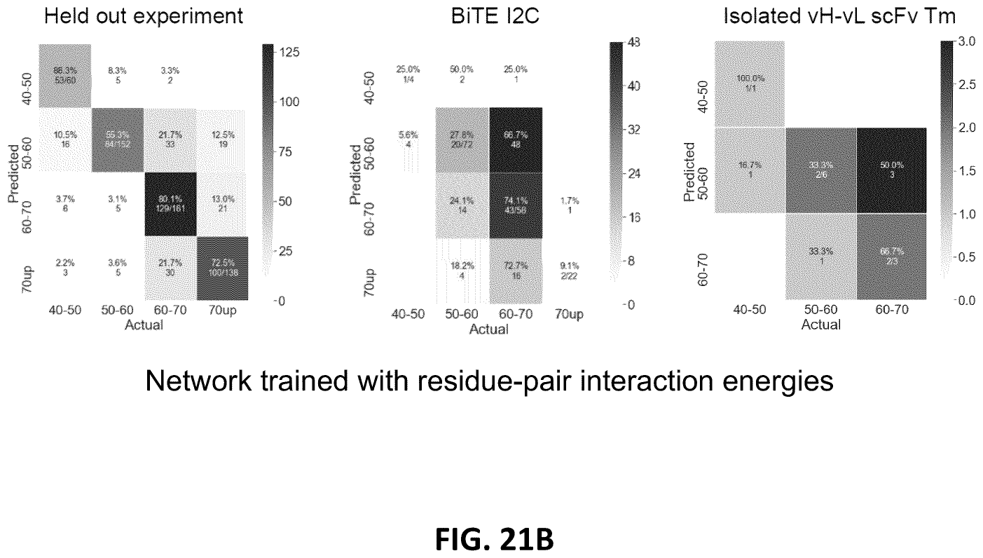

FIGS. 21A and 21B are graphs showing that training and prediction based on residue-pair interaction energy metrics is more accurate than training and prediction based on residue-pair interaction energy metrics and encoded sequences, in accordance with some embodiments of the technology described herein.

D. Datasets

To learn temperature-specific contextual patterns in sequence-data, machine learning models were developed and trained for thermostability prediction using scFv sequences. Temperature data was collected from various antibody engineering studies for developing thermostable scFv antibodies. The sequence data contained scFv sequences assembled by performing mutations to heavy and light chains from multiple germlines. 2,700 scFv sequences from 17 germlines (further referred as experimental sets) were collated to constitute the sequence data. Additionally, sequences from another scFv study (currently under trials) and an isolated scFv dataset form blind test sets.

For each sequence, thermostability is evaluated with a TS50 measurement representing the temperature at half-maxima of target binding, and this measurement serves as the temperature annotation. The TS50 data may also be divided into four classes. For example, the TS50 data may be divided up into under-50° C., 50° C.-60° C., 60° C.-70° C., and 70° C.-up.

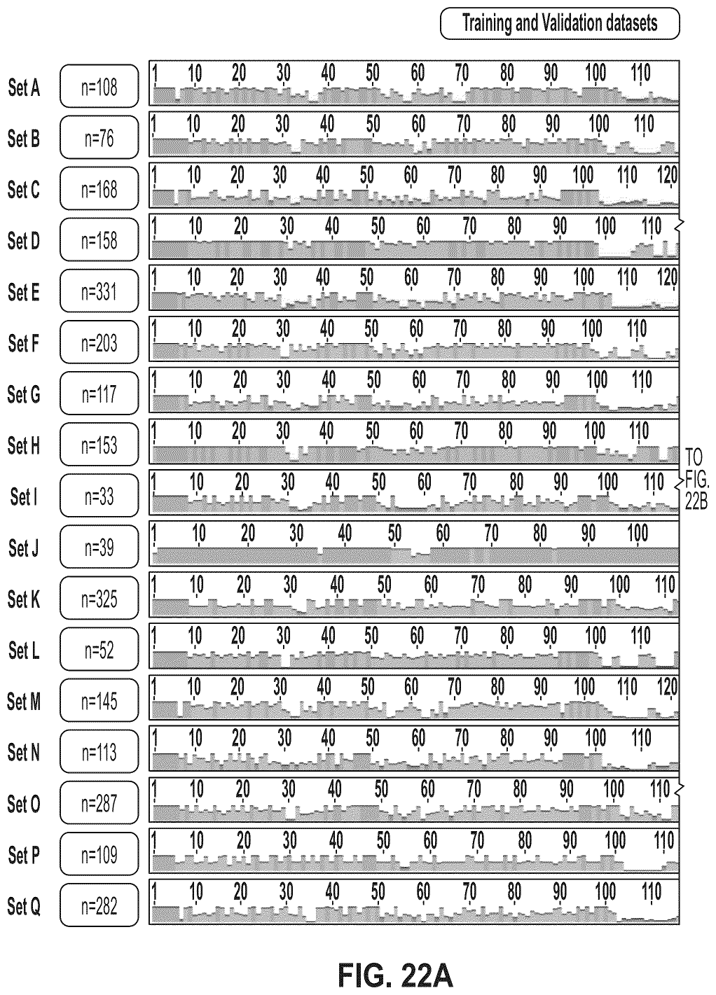

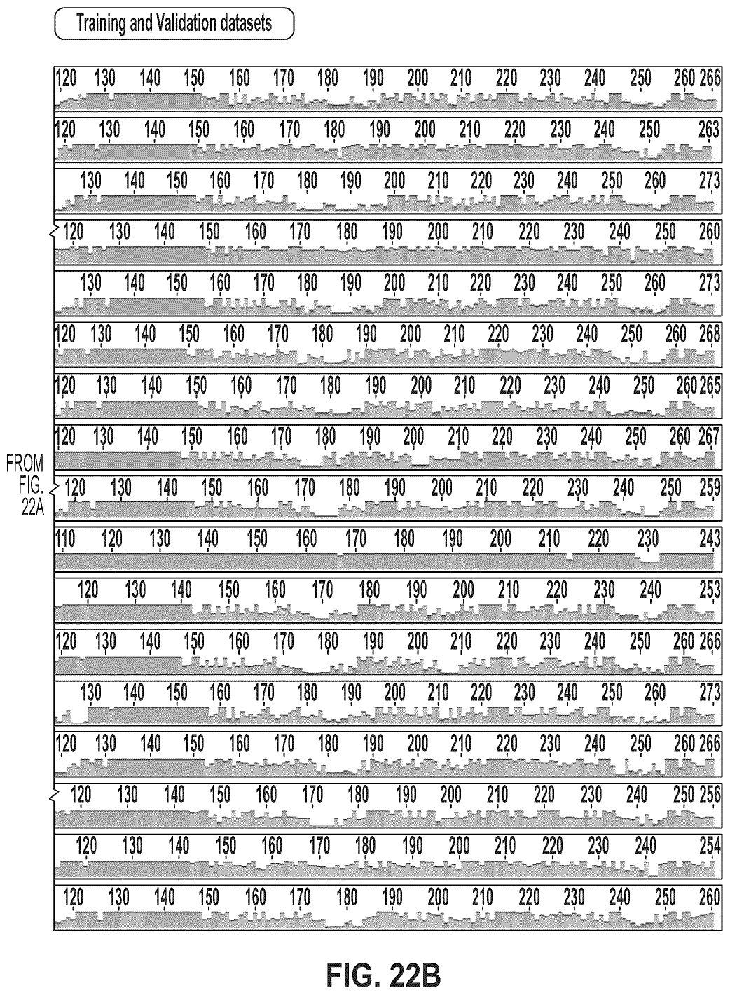

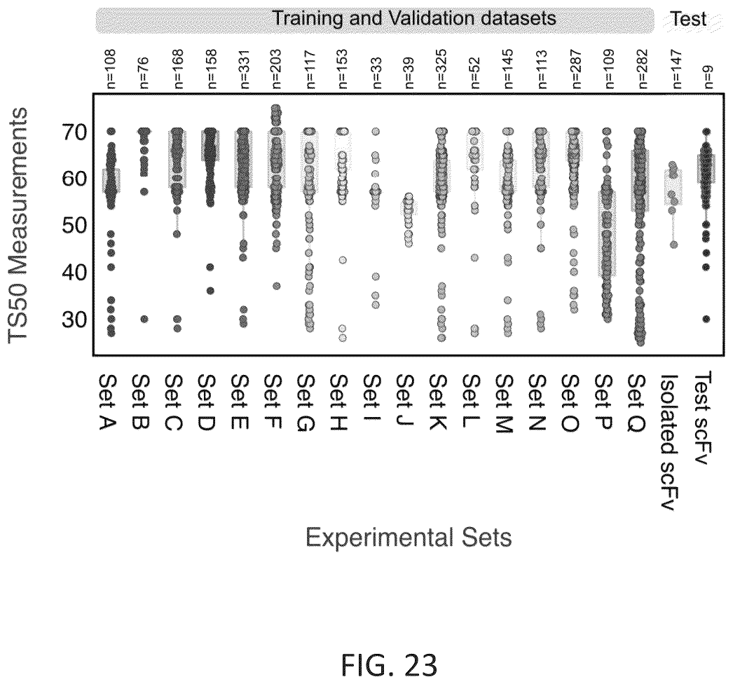

The experimental dataset was non-uniform and potentially skewed towards the higher temperature classes (i.e., 60° C.-70° C. and 70° C.-up classes). A distribution of the training, validation and test datasets is shown in FIGS. 22A-22B. The sequence data representation is such that the taller bars represent greater consensus. The Gly₄/Ser linker region between the V_{H} (heavy) and V_{L} (light) chains is evident. For machine learning model training, the heavy and light chain sequences were separated from the linker. FIG. 23 highlights the temperature distribution of the TS50 measurements to show the skewed nature of the experimental dataset.

i. Generation of scFvs

scFvs with a (G4S)3 linker were cloned as a single construct into a pTT vector with a puromycin selection marker. Constructs were transfected into a mammalian CHO-K1 cell line and stably expressed at a 4 mL scale. After 21 days post transfection, VCD and viability were measured and the expression level of secreted proteins in conditioned medium were analyzed by non-reduced SDS PAGE gel. Cells were further incubated with magnetic beads coupled with either proA (for scFvs with lambda variable domains) or proL (for scFvs with kappa variable domains) overnight. The beads were separated from cell media and following by washed with PBS for three times and water for 2 times. scFvs were eluted from the magnetic beads with a low pH buffer (100 mM glycine, pH2.7) and neutralized with 3M Tris (pH11). Differential Scanning Fluorimetry (DSF) was carried out to determine the melting temperature of the purified material. Briefly, molecules were heated at 1.0° C./min on a nanoDSF instrument. Changes in tryptophan fluorescence were monitored to evaluate protein unfolding and aggregation. The Tₘ is reported as the midpoint between the unfolding onset and the max unfolded state.

ii. TS50 Screening Assay

The thermostability of scFvs was screened by determining the loss of target binding after high temperature stress. To this end, soluble scFvs (VH-(G4S)3-VL) containing a C-terminal FLAG-tag (DYKDDDDK) and a 6xHis-tag were produced in E. coli TG1 (Agilent, Santa Clara, USA) in 10 mL LB cultures. Protein production was induced with 1 mM IPTG. Bacteria were then centrifuged, and the cell pellet was resuspended in 1 mL Gibco™ DPBS. Cells were lysed with 4 freeze/thaw cycles and residual cells and cell debris were removed by two centrifugation steps. 100μL of these crude extracts were transferred into 0.2 mL tubes and subjected for 5 min to different temperatures in water baths (4° C., 50° C., 60° C., 70° C.). After incubation, the tubes were directly transferred on ice and human target transfected CHO-cells were incubated with 50 μL of the lysates. Bound scFvs were detected and analyzed by flow cytometry. Median fluorescence intensity values were determined and plotted. The temperature corresponding to half maximal binding of each scFv was calculated (TS50). The scFv sequences were further classified into sets based on the identity of the antigen they bind (not random). Since the sets were not curated for a machine learning task, there is lack of a uniform distribution across sets.

iii. nanoDSFTₘ Method

Thermal melting (Tₘ) temperatures were determined by running a Trp Shift Study on the Prometheus, NT.48. A thermal ramp was applied at 1.0° C./min with start temperature 25° C. and stop temperature with 95° C. Unfolding was measured by the fluorescence ratio 350 nm/330 nm. Data analysis and Tₘ determination was performed using PR. ThermControl v2.0.4. Samples were normalized to 1.0 mg/mL in formulation buffer prior to Tm analysis.

iv. Dataset Curation for Supervised Models

To generate sequence inputs, datasets for TS50 measurements of scFvs from all experimental sets were aggregated to form a single dataset. The scFv sequences comprised of a heavy and a light chain linked together with a Glycine-Serine (G4S)ₓ linker. The dataset was created with the scFv sequences, split into their respective heavy and light chain sequences, and instead of classifying sequences based on their thermostability, their TS50 measurements were included. The distribution of the scFv sequences across the experimental sets and the test dataset is illustrated in FIG. 23. Sets P and Q were removed, along with the sequences of the test antibody and the isolated scFv to constitute the held-out set. The amino acid sequences were one-hot encoded to form an input of dimension, (V_{H}+V_{L}+3)×21, where V_{H} and V_{L} correspond to the heavy and light chain sequences, respectively. The additional token to the amino acids' one-hot encoding corresponds to the delimiter at the start and end positions of the scFv sequence, and between heavy and light chains to indicate a chain break.

To obtain the energetics input, the sequences were first passed through a structural module i.e., the DeepAb protocol for protein structure prediction. A Rosetta Relax and refinement protocol for side-chain repacking (XML scripts in the Supplementary) was run for each predicted structure. Energy estimation in Rosetta starts with an energy relaxation step to reduce steric clashes (Rosetta Relax) with constraints to the start coordinates so that the accuracy of backbone structure (predicted by DeepAb) is not diminished. The all-atom model is refined further with 4 cycles of side-chain packing to obtain a robust structure and the lowest energy structure is chosen for further calculations. For each refined model, the residue-residue interaction energy metrics were estimated with the residue energy breakdown application. The one-body and two-body energies were converted to a two-dimensional i-j matrix that served as the energetic information for training in the supervised CNN models.

The energy values in the i-j matrix were also further classified together in 20 classes between the lower-end and upper-end energies of 1-25,101 REU, respectively. An additional class was included for the start, end, and chain-break tokens, respectively. The dimension of the pairwise energy data is this, L×L×21.

An illustrative implementation of a computer system 2400 that may be used in connection with any of the embodiments of the technology described herein (e.g., such as the methods of FIGS. 2A-2B, FIGS. 4A-4B, and FIG. 6) is shown in FIG. 24. The computer system 2400 includes one or more processors 2410 and one or more articles of manufacture that comprise non-transitory computer-readable storage media (e.g., memory 2420 and one or more non-volatile storage media 2430). The processor 2410 may control writing data to and reading data from the memory 2420 and the non-volatile storage media 2430 in any suitable manner, as the aspects of the technology described herein are not limited to any particular techniques for writing or reading data. To perform any of the functionality described herein, the processor 2410 may execute one or more processor-executable instructions stored in one or more non-transitory computer-readable storage media (e.g., the memory 2420), which may serve as non-transitory computer-readable storage media storing processor-executable instructions for execution by the processor 2410.

Computer system 2400 may also include a network input/output (I/O) interface 2440 via which the computing device may communicate with other computing devices (e.g., over a network), and may also include one or more user I/O interfaces 2450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

1. A method for computationally screening a set of single-chain variable fragments (scFvs) based on thermostability of the scFvs predicted by a trained machine learning model, the set of scFvs comprising scFvs having different residue sequences, the method comprising:
determining, using the trained machine learning model and at least one computer hardware processor, a thermostability indication for each scFv in the set of scFvs to obtain a plurality of thermostability indications, the set of scFvs comprising a first scFv having a first residue sequence, the determining comprising:
obtaining, using information indicative of a three-dimensional (3D) structure of the first scFv, interaction energy metrics for each of a plurality of pairs of residues, the residues being in the first residue sequence;
generating a first set of features to provide as input to the trained machine learning model, the generating comprising including the interaction energy metrics in the first set of features; and
providing the first set of features as input to the trained machine learning model to obtain a corresponding output indicative of a first thermostability for the first scFv;
identifying a subset of the set of scFvs for subsequent production based on the plurality of thermostability indications; and
producing at least one of the scFvs in the identified subset.

2. The method of claim 1, wherein the set of scFvs further comprises a second scFv different from the first scFv, the second scFv having a second residue sequence, and wherein determining the thermostability indication for each scFv in the set of scFvs further comprises:
obtaining second interaction energy metrics for each of a second plurality of pairs of second residues, the second plurality of pairs of second residues being in the second residue sequence;
generating a second set of features to provide as input to the trained machine learning model, the generating comprising including the second interaction energy metrics in the second set of features; and
providing the second set of features as input to the trained machine learning model to obtain a corresponding output indicative of a second thermostability for the second scFv.

3. The method of claim 1, wherein the output indicative of the first thermostability for the first scFv indicates a first temperature at which the first scFv is thermostable.

4. The method of claim 3, wherein the first temperature is an estimate of a temperature corresponding to half maximal binding of the first scFv.

5. The method of claim 1, wherein the output indicative of the first thermostability for the first scFv indicates a first temperature range including at least one temperature at which the first scFv is thermostable.

6. The method of claim 5, wherein the first temperature range is an estimate of a temperature range that includes a temperature corresponding to half maximal binding of the first scFv.

7. The method of claim 1, wherein providing the first set of features as input to the trained machine learning model to obtain the output indicative of the first thermostability for the first scFv comprises:
classifying, using the trained machine learning model, the first scFv into one of a plurality of classes using the first set of features, wherein each of the plurality of classes corresponds to a respective temperature range.

8. The method of claim 1, wherein obtaining the interaction energy metrics comprises:
determining the information indicative of the 3D structure of the first scFv by using protein structure prediction software to generate the information indicative of the 3D structure from the first residue sequence.

9. The method of claim 8, wherein obtaining the interaction energy metrics comprises:
determining the interaction energy metrics using molecular modeling software to generate the interaction energy metrics using the information indicative of the 3D structure of the first scFv.

10. The method of claim 1, wherein generating the first set of features comprises:
for each particular energy metric of the interaction energy metrics,
generating a respective two-dimensional (2D) matrix of values of the particular energy metric, wherein rows and columns of the 2D matrix correspond to respective residues in the first residue sequence, and wherein an entry in an ith row and jth column of the 2D matrix corresponds to a value of the particular energy metric for an ith residue in the first residue sequence and a jth residue in the first residue sequence; and
including the generated 2D matrix in the first set of features.

11. The method of claim 10, wherein the generated 2D matrix includes a row for at least 75% of the residues in the first residue sequence.

12-15. (canceled)

16. The method of claim 1, wherein generating the first set of features further comprises:
encoding the first residue sequence to obtain an encoded sequence; and
including the encoded sequence in the first set of features.

17. (canceled)

18. The method of claim 1, wherein the trained machine learning model comprises a trained neural network model.

19. The method of claim 18, wherein the trained neural network model comprises a trained convolutional neural network (CNN) model, the trained CNN model having a plurality of 2D convolutional layers.

20. (canceled)

21. The method of claim 19, wherein the trained CNN model is configured to output a plurality of probabilities that an scFv is thermostable in each of a plurality of temperature ranges.

22. The method of claim 21, wherein providing the first set of features as input to the trained machine learning model to obtain the corresponding output indicative of the first thermostability for the first scFv comprises:
providing the first set of features to the trained CNN model to obtain a first plurality of probabilities that the first scFv is thermostable in each of the plurality of temperature ranges; and
determining the first thermostability as either:
(i) a temperature range in the plurality of temperature ranges associated with a highest probability in the first plurality of probabilities; or
(ii) a temperature determined as a weighted linear combination of mean values of the plurality of temperature ranges weighted by the probabilities in the first plurality of probabilities.

23. The method of claim 1, wherein identifying the subset of the set of scFvs for subsequent production based on the determined thermostability indications comprises:
determining whether the first thermostability for the first scFv satisfies at least one criterion; and
after determining that the first thermostability satisfies the at least one criterion, identifying the first scFv for subsequent production.

24. The method of claim 1, further comprising: testing the thermostability of the at least one of the scFvs in an in vitro assay.

25-31. (canceled)

32. A system, comprising:
at least one computer hardware processor; and
at least one non-transitory computer-readable storage medium storing processor-executable instructions that, when executed by the at least one computer hardware processor, cause the at least one computer hardware processor to perform a method for computationally screening a set of single-chain variable fragments (scFvs) based on thermostability of the scFvs predicted by a trained machine learning model, the set of scFvs comprising scFvs having different residue sequences, the method comprising:
determining, using the trained machine learning model and at least one computer hardware processor, a thermostability indication for each scFv in the set of scFvs to obtain a plurality of thermostability indications, the set of scFvs comprising a first scFv having a first residue sequence, the determining comprising:
obtaining, using information indicative of a three-dimensional (3D) structure of the first scFv, interaction energy metrics for each of a plurality of pairs of residues, the residues being in the first residue sequence;
generating a first set of features to provide as input to the trained machine learning model, the generating comprising including the interaction energy metrics in the first set of features; and
providing the first set of features as input to the trained machine learning model to obtain a corresponding output indicative of a first thermostability for the first scFv;
identifying a subset of the set of scFvs for subsequent production based on the plurality of thermostability indications; and
producing at least one of the scFvs in the identified subset.

33. At least one non-transitory computer-readable storage medium storing processor-executable instructions that, when executed by at least one computer hardware processor, cause the at least one computer hardware processor to perform a method for computationally screening a set of single-chain variable fragments (scFvs) based on thermostability of the scFvs predicted by a trained machine learning model, the set of scFvs comprising scFvs having different residue sequences, the method comprising:
determining, using the trained machine learning model and at least one computer hardware processor, a thermostability indication for each scFv in the set of scFvs to obtain a plurality of thermostability indications, the set of scFvs comprising a first scFv having a first residue sequence, the determining comprising:
obtaining, using information indicative of a three-dimensional (3D) structure of the first scFv, interaction energy metrics for each of a plurality of pairs of residues, the residues being in the first residue sequence;
generating a first set of features to provide as input to the trained machine learning model, the generating comprising including the interaction energy metrics in the first set of features; and
providing the first set of features as input to the trained machine learning model to obtain a corresponding output indicative of a first thermostability for the first scFv;
identifying a subset of the set of scFvs for subsequent production based on the plurality of thermostability indications; and
producing at least one of the scFvs in the identified subset.
